(19) United States
(12) Patent Application Publication
(10) Pub. No.: US 2025/0381205 A1
(43) Pub. Date: Dec. 18, 2025

(54) Transdermal Delivery of Dronabinol

(71) Applicant: Pike Therapeutics USA, Inc., Wilmington, DE (US)

(72) Inventors: Fotios M. Plakogiannis, Whitestone, NY (US); Tamanna Lather, Jersey City, NJ (US); Nisarg Modi, Jersey City, NJ (US); Marina Borovinskaya, East Brunswick, NJ (US)

(73) Assignee: Pike Therapeutics USA, Inc., Wilmington, DE (US)

(21) Appl. No.: 19/302,651

(22) Filed: Aug. 18, 2025

Related U.S. Application Data
(63) Continuation-in-part of application No. 17/225,038, filed on Apr. 7, 2021, now Pat. No. 12,409,131.
Application No. 17/225,038 is a continuation-in-part of application No. 17/221,667, filed on Apr. 2, 2021, now Pat. No. 12,409,132.
Application No. 17/221,667 is a continuation-in-part of application No. PCT/US2020/054070, filed on Oct. 2, 2020.
(60) Provisional application No. 62/910,255, filed on Oct. 3, 2019.

(51) Int. Cl. A61K 31/00; A61K 9/00; A61K 9/70; A61K 47/10; A61K 47/32

(52) U.S. Cl. CPC A61K 31/658; A61K 9/0021; A61K 9/7084; A61K 47/10; A61K 47/32

(57) ABSTRACT

Provided is a transdermal drug delivery system comprising dronabinol. The dronabinol transdermal delivery system provides a drug plasma concentration at predetermined rate for a predetermined period of time, offering a simplified therapeutic regimen by decreasing dosing frequency for the treatment and/or prevention of nausea and/or vomiting associated with, for example, chemotherapy.

### CROSS-REFERENCE TO RELATED APPLICATIONS

This application is a continuation in part of U.S. patent application Ser. No. 17/225,038, filed Apr. 7, 2021, which is a continuation-in-part of, and claims the benefit of and priority to, U.S. patent application Ser. No. 17/221,667, filed on Apr. 2, 2021, which is a continuation-in-part of, and claims the benefit of and priority to, International Application No. PCT/US2020/054070, filed on Oct. 2, 2020, which claims the benefit of U.S. Provisional Application No. 62/910,255, filed Oct. 3, 2019, all of which are incorporated by reference herein in their entirety for all purposes.

### TECHNICAL FIELD

The present disclosure relates to compositions and methods for treating or controlling a psychological or neurological disease or disorder in a patient in need thereof. The present disclosure relates to treatment of nausea and vomiting, such as nausea and vomiting commonly experienced by patients undergoing chemotherapy.

### BACKGROUND

Dementia is not a specific disease, but a condition that is associated with a wide range of symptoms that, in turn, are associated with a decline in memory, among other brain functions. Dementia can affect the young, middle aged, and elderly, but it is generally more prevalent in adults over the age of 60. More than three million cases of dementia are diagnosed yearly in the United States alone.

It is believed that dementia results from damage to nerve cells in the brain, brain cell death, and/or neurodegenerative diseases. Some dementias are believed to be due to exogenous factors such as mediations, deficiencies, such as vitamin deficiencies, head injuries, strokes, brain tumors, prion diseases, and HIV infection. However, no single one reason why a patient develops dementia has been identified.

Dementia can affect different areas of the brain, thereby resulting in different symptoms. The most common symptom of dementia is memory loss, which may be short term or long term, and greatly affects quality of life for patients of all ages. Although dementia generally cannot be cured, there are medications and therapies that can delay halt or even delay the progression of symptoms. There is a need for effective methods of treating, preventing, controlling, and/or ameliorating dementia.

Alzheimer's disease, the most common age-related dementia, is a devastating neurological disorder currently afflicting more than 5.5 million Americans at costs that exceed $200 billion per year. AD is characterized by the presence of senile plaques largely comprising amyloid-β peptide, and neurofibrillary tangles resulting from hyperphosphorylation of tau (p-tau), in brain tissue. AD patients suffer from deficits in cognition, learning, and memory; and have impaired long-term potentiation as well as disruption in cholinergic neurotransmission. Only two types of drugs are currently approved for the treatment of AD: acetylcholinesterase (AChE) inhibitors and N-methyl-D-aspartyl (NMDA) receptor antagonists; the former enhance activity of the neurotransmitter acetylcholine and the latter reduce excitotoxicity resulting from NMDA receptor over-activation. Both types of drugs only provide temporary, symptomatic relief and a modest delay in cognitive decline in patients, with beneficial effects typically only being maintained up to 36 months. Identification of the Aβ peptide, cloning of the amyloid precursor protein gene, and findings by many that specific mutations in APP result in familial early-onset AD led to the establishment of the Amyloid Cascade Hypothesis of AD. This hypothesis suggests that it is the over-production of Aβ, or reduced clearance, that drives the development and progression of the disease. Aβ is derived from processing of full-length APP, whereby sequential cleavage by β-secretase BACE1 (BACE) and the γ-secretase complex produces first soluble APPB (sAPPB) and the b-C-terminal fragment (BCTF), and then (from BCTF) Aβ of a variety of lengths (species). Alternatively, α-secretase cleavage (putatively by ADAM10) results in production of trophic, synapse-supporting peptides soluble APPα (sAPPα) and the α-C-terminal fragment (αCTF).

Motor neuron diseases are a class of neurological diseases that result in the degeneration and death of motor neurons—those neurons which coordinate voluntary movement of muscles by the brain. Motor neuron diseases may be sporadic or inherited, and may affect upper motor neurons and/or lower motor neurons. Motor neuron diseases include amyotrophic lateral sclerosis, progressive bulbar palsy, pseudobulbar palsy, primary lateral sclerosis, progressive muscular atrophy, spinal muscular atrophy, and post-polio syndrome.

Amyotrophic lateral sclerosis (ALS) is a group of motor neuron diseases affecting about 15,000 individuals in the United States of America. ALS is characterized by degeneration and death of upper and lower motor neurons, resulting in loss of voluntary muscle control. Motor neuron death is accompanied by muscle fasciculation and atrophy. Early symptoms of ALS include muscle cramps, muscle spasticity, muscle weakness (for example, affecting an arm, a leg, neck, or diaphragm), slurred and nasal speech, and difficulty chewing or swallowing. Loss of strength and control over movements, including those necessary for speech, eating, and breathing, eventually occur. Disease progression may be accompanied by weight loss, malnourishment, anxiety, depression, increased risk of pneumonia, muscle cramps, neuropathy, and possibly dementia. Most individuals diagnosed with ALS die of respiratory failure within five years of the first appearance of symptoms. Currently, there is no effective treatment for ALS.

ALS occurs in individuals of all ages, but is most common in individuals between 55 to 75 years of age, with a slightly higher incidence in males. ALS can be characterized as sporadic or familial. Sporadic ALS appears to occur at random and accounts for more than 90% of all incidences of ALS. Familial ALS accounts for 5-10% of all incidences of ALS.

Frontotemporal dementia (FTD) refers to a spectrum of progressive neurodegenerative diseases caused by loss of neurons in frontal and temporal lobes of the brain. FTD is the third most common form of dementia (following Alzheimer's disease and dementia with Lewy bodies), and the second most common form of dementia in individuals below 65 years of age. FTD is estimated to affect 20,000 to 30,000 individuals in the United States of America. FTD is characterized by changes in behavior and personality, and language dysfunction. Forms of FTD include behavioral variant FTD (bvFTD), semantic variant primary progressive aphasia (svPPA), and nonfluent variant primary progressive aphasia (nfvPPA). ALS with FTD is characterized by symptoms associated with FTD, along with symptoms of ALS such as muscle weakness, atrophy, fasciculation, spasticity, speech impairment (dysarthria), and inability to swallow (dysphagia). Individuals usually succumb to FTD within 5 to 10 years, while ALS with FTD often results in death within 2 to 3 years of the first disease symptoms appearing. Like ALS, there is no known cure for FTD, or ALS with FTD, nor a therapeutic known to prevent or retard either disease's progression.

The present disclosure relates to pharmaceutical compositions and methods for the reduction of seizure frequency in the treatment of, for example, “treatment-resistant epilepsy” (TRE), including treatment resistant pediatric epilepsy, or Tuberous Sclerosis Complex (TSC), Dravet Syndrome and Lennox-Gastaut Syndrome. In one embodiment the patients suffering from TRE are children and young adults. Lennox-Gastaut Syndrome (LGS) is a severe form of epilepsy that typically becomes apparent during infancy or early childhood. Onset of LGS is usually between 2-7 years with a peak onset between 3 to 5 years. Affected Children experience several different types of seizures most commonly atonic, tonic and atypical absence seizures.

Anxiety, depression, and stress are symptoms of psychological disorders and are the most prevalent psychiatric symptoms, being associated with a high burden of illness. For example, up to one-third of women and one-fifth of men will experience anxiety at some point in their lives. Insomnia is a further significant health burden, often associated with psychological disorder. Agitation is a state of heightened psychomotor activity often accompanied by emotional distress, irritability, or aggression. It is characterized by restlessness, pacing, excessive talking, shouting, physical outbursts, or other disruptive behaviors. Agitation is frequently observed in patients with neurodegenerative disorders, traumatic brain injuries, psychiatric conditions, and metabolic or systemic illnesses. It can significantly impair quality of life, interfere with care, and pose safety risks to both the affected individual and caregivers. Current therapeutic approaches include sedative medications, antipsychotics, mood stabilizers, and behavioral interventions, yet these options often provide incomplete symptom control and may be associated with undesirable side effects, including sedation, cognitive impairment, or increased risk of falls.

Chronic Traumatic Encephalopathy (CTE) is a progressive neurodegenerative disease associated with repetitive head trauma, such as that experienced by contact sports athletes, military veterans, and others exposed to repeated concussive or subconcussive impacts. CTE is characterized by the accumulation of hyperphosphorylated tau protein in specific brain regions, leading to cognitive impairment, mood disturbances, impulsivity, and behavioral dysregulation. Agitation is a common and distressing behavioral manifestation in individuals with CTE, often presenting alongside aggression, emotional lability, and poor impulse control. The management of agitation in CTE poses unique challenges, as patients may have comorbid cognitive decline, psychiatric symptoms, and heightened sensitivity to adverse effects of standard psychotropic medications. There remains a need for safe, effective, and well-tolerated therapies for the treatment of agitation, particularly agitation associated with CTE and other neurodegenerative or trauma-related conditions.

The present disclosure provides a composition and method to treat, reduce, or prevent symptoms related to agitation, agitation associated with CTE, anxiety, stress, depression, and insomnia, and/or to provide the consumer with a useful or commercial choice.

There is a pressing need to for compounds and/or compositions capable of treating, preventing, controlling, and/or ameliorating neurological diseases such as: dementia, amyotrophic lateral sclerosis (ALS), frontotemporal dementia (FTD), dementia, ALS with FTD, Alzheimer's disease (AD), Parkinson's disease (PD), Huntington's disease, progressive supranuclear palsy (PSP), brain trauma, spinal cord injury, corticobasal degeneration (CBD), seizure, agitation, agitation associated with CTE, anxiety, stress, depression, insomnia, nerve injuries (e.g., brachial plexus injuries), neuropathies (e.g., chemotherapy induced neuropathy), and TDP43 proteinopathies (e.g., chronic traumatic encephalopathy, Perry Syndrome, Dementia with Lewy body in association with Alzheimer's disease, Parkinson's disease with or without dementia, and Limbic-predominant age-related TDP-43 encephalopathy (LATE)).

Accordingly, disclosed herein are methods of treating a neurological disease or disorder in a patient in need thereof, the method comprising administering to a patient in need thereof a therapeutically effective amount of a pharmaceutical composition disclosed herein. In various embodiments, the neurological disease is selected from the group consisting of dementia, amyotrophic lateral sclerosis (ALS), frontotemporal dementia (FTD), ALS with FTD, Alzheimer's disease (AD), Parkinson's disease (PD), Huntington's disease, progressive supranuclear palsy (PSP), brain trauma, spinal cord injury, corticobasal degeneration (CBD), nerve injuries (e.g., brachial plexus injuries), neuropathies (e.g., chemotherapy induced neuropathy), and TDP43 proteinopathies (e.g., chronic traumatic encephalopathy, Perry Syndrome, Dementia with Lewy body in association with Alzheimer's disease, Parkinson's disease with or without dementia, and Limbic-predominant age-related TDP-43 encephalopathy (LATE)). In various embodiments, the neurological disease is ALS. In various embodiments, the neurological disease is FTD. In various embodiments, the neurological disease is ALS with FTD. In various embodiments, the neuropathy is chemotherapy induced neuropathy. In various embodiments, the pharmaceutical composition is administered topically, parenterally, orally, pulmonarily, rectally, buccally, sublingually, vaginally, intratracheally, intranasally, intracisternally, intrathecally, intrathalamically, intravenously, intramuscularly, transdermally, or intraduodenally. In various embodiments, wherein the pharmaceutical composition is administered intrathecally, intrathalamically intracerebroventricularly, or intracisternally. In various embodiments, a therapeutically effective amount of the oligonucleotide is administered intrathecally, intrathalamically or intracisternally. In various embodiments, the patient is human.

Nausea and vomiting are the common side effects of cancer chemotherapy. Drugs such as dronabinol and 5-HT₃ receptor antagonists are recommended for the treatment of nausea and vomiting associated with cancer chemotherapy. At times, to prevent nausea and vomiting one or more drugs can be used (See Herrsted J., et al., 2016 Updated MASCC/ESMO Consensus Recommendations: Prevention of Nausea and Vomiting Following High Emetic Risk Chemotherapy. Support Care Cancer. 2017 January; 25 (1): 277-288. Epub 2016 Jul. 22).

Dronabinol is a synthetic form of delta-9-tetrahydrocannbidiol (delta-9-THC) in sesame oil. Dronabinol is approved for use in treating emesis, and is available as an oral capsule (MARINOL) and as an oral solution (SYNDROS). As stated in the package insert or drug label for the oral capsule of dronabinol the drug “is indicated for the treatment of nausea and vomiting associated with cancer chemotherapy in patients who have failed to respond adequately to conventional antiemetic treatments. It is also indicated for the treatment of anorexia associated with weight loss in patients with AIDS” (See Label for NDA Marinol, Labeling, Label action date Jun. 21, 2006 accessed on Jul. 13, 2017, hereafter “Label”).

Many shortcomings are associated with the dronabinol oral capsule. The first challenge is the dosage regimen. For an antiemetic effect the following dosage regimen is recommended: 1 to 3 hours before the chemotherapy a first dose of 5 mg/m² is administered orally. After chemotherapy drug is administered every 2 to 4 hours in total 4 to 6 doses per day. Depending on the clinical response, if the dose is not sufficiently effective then the dose is increased by increments of 2.5 mg/m² up to about 15 mg/m². This dosage regimen is inconvenient for patients who are already experiencing nausea and vomiting. A second challenge is that the pharmacologic response is dose related and there is interpatient variability. A third challenge is that at maximum dose the likelihood of disturbing psychiatric symptoms increases. A fourth challenge is that after oral administration dronabinol undergoes first pass hepatic metabolism and has high lipid solubility; therefore, of the administered dose 10%-20% reaches systemic circulation. Another challenge is that at room temperature the active ingredient dronabinol is unstable is the capsules, and so the capsules are packaged in a closed container and recommended storage is in refrigerator or between 8° C.-15° C. (See FDA Label www.accessdata.fda.gov/drugsatfda_docs/label/2017/018651s0291bl.pdf). The dronabinol capsule is stable for only 3 months at room temperature. This is due to synthetic delta-9-THC exhibiting rapid oxidation and acid and basic degradation. Moreover, synthetic delta-9-THC also degrades more rapidly in light and at higher temperatures the degradation rate increases. Accordingly, if dronabinol capsules are not stored refrigerated and in the original container, the concentration of delta-9-THC in the capsules will decrease, potentially below the therapeutically required concentration.

Dronabinol is a synthetic form of delta-9-tetrahydrocannbidiol (delta-9-THC) in sesame oil. Previous studies have been performed with naturally derived delta-9-THC from marijuana plant cannabis Sativa L. However, it is difficult to extract pure delta-9-THC from a plant source, such as Sativa L. This is caused by an adulteration of the extract by small amounts of another active cannabidiols (such as cannabinol, delta-8-THC, cannabidiol and cannabichromene) present in final product. Accordingly, the amount of THC in final extract is dependent on the extraction process which is important due to the psychoactive properties of THC, among other considerations. Alternatively, the synthetic form of delta-9-THC is developed under a more controlled procedure as opposed to the extraction process for its naturally derived counterpart. For example, synthetically produced delta-9-THC consists essentially of uncontaminated delta-9-THC that is not adulterated by the presence of other active cannabidiols. Therefore, the synthetic version of delta-9-THC is able to provide improved transdermal permeability as compared to previously published work with naturally derived delta-9-THC which demonstrates a maximum flux of 0.01 μg/cm²/hr through rat skin for naturally extracted delta-9-THC (U.S. Pat. No. 6,503,532).

U.S. Pat. No. 6,328,992 discloses the preparation of transdermal delivery of cannabinoids. However, the disclosure describes different cannabinoids in combination and does not describe the use of pure synthetic delta-9-THC alone. Furthermore, the '992 patent inventors describe the use of permeability enhancers which are not pharmaceutical grade and not included in U.S. Food and Drug Administration Inactive Ingredient Listing (FDA IIG). Therefore the disclosure of the '992 does not provide a composition with any pharmaceutical utility. Furthermore, the '992 patent describes the use of rat skin with 50% ethanol in receiving media for the transdermal permeability testing. However, ethanol destroys the dermis skin structure and can increase the in-vitro flux value thus providing results that are not germane to transdermal patches for pharmaceutical applications. For example, ethanol treated rat skin is almost ten time more permeable than human cadaver skin.

U.S. Pat. No. 8,449,908 provides delivery of the cumulative amount of 10000 ng THC in 96 hrs through the human cadaver skin. This amount represents the flux of 60 ng/cm²/hr. The patch area can be calculated using following equation:

$$\begin{matrix}In - Vitro Flux ( ng / {cm}^{2} / hr ) = ( {C}_{SS} ( ng / mL ) * CL ( L / kg * hr ) * BW ( Kg ) ) / Patch Area ( {cm}^{2} ) \\ Patch Area ( {cm}^{2} ) = ( Css ( ng / mL ) * CL ( L / kg * hr ) * BW ( kg ) ) / In - Vitro Flux ( ng / {cm}^{2} / hr ) \\ = ( 1.38 * 0.2 * 70 * 1000 ) / 60 \\ = 322 {cm}^{2}\end{matrix}$$

In order to deliver a therapeutic dosage of 5 mg/day of delta-9-THC, a transdermal formulation would need to cover at least 325 cm² surface area of the patient's skin. This is an impractical patch size for any transdermal drug delivery system (TDDS).

Therefore, there is a need for an improved drug delivery system of dronabinol which can overcome above stated drawbacks associated with oral administration and naturally derived delta-9-THC. As provided herein, transdermal delivery of dronabinol comprising synthetic delta-9-THC can address the challenges associated with oral drug delivery.

### BRIEF SUMMARY

The following aspects and embodiments thereof described and illustrated below are meant to be exemplary and illustrative, not limiting in scope.

In one aspect, a pharmaceutical composition comprising dronabinol in a dosage form for transdermal delivery is provided.

In an embodiment, the pharmaceutical composition comprises no additional antiemetic drug.

In an embodiment, dronabinol is in a form selected from the group consisting of co-crystals, amorphous, coated, crystalline, a salt, an isomer, a solid solution, a prodrug, an analog, a derivative, a metabolite, a solution, synthetic, an ethanol solution, sesame oil solution, and a naturally derived delta-9-tetrahydrocannabinol.

In an embodiment, dronabinol is in the composition at between about 0.01%-95% w/w or between about 0.01%-95% w/v.

In an embodiment, dronabinol is selected from a group consisting of amorphous dronabinol, crystalline dronabinol, co-crystals of dronabinol, coated dronabinol, and ethanolic solution of dronabinol in the range of 0.01%-95% w/w or w/v.

In an embodiment, dronabinol is in a salt form.

In an embodiment, the composition is formulated as transdermal liquid formulation, transdermal semisolid formulation and/or transdermal polymer matrix formulation.

In an embodiment, a carrier or an ingredient in effective amount either alone or in combinations thereof is included in the composition. In an embodiment, the carrier or ingredient is selected from the group consisting of solvents, gelling agents, polymers, penetration enhancers, emollients, skin irritation reducing agents, buffering agents, pH stabilizers, solubilizers, suspending agents, dispersing agents, stabilizers, plasticizers, surfactants, antioxidants, and oxidants.

In an embodiment, the carrier or ingredient is in the composition in a range of between about 0.01%-95% w/w or w/v.

In an embodiment, the pharmaceutical composition is formulated as a transdermal patch.

In an embodiment, the transdermal patch is selected from the group consisting of a reservoir patch, a micro-reservoir patch, a matrix patch, a pressure sensitive adhesive patch, and an extended release transdermal film.

In an embodiment, the pharmaceutical composition is formulated as microneedles.

In an embodiment, the microneedles are formulated as a transdermal patch.

In an embodiment, a method for the treatment and/or prevention and/or control of nausea and/or vomiting associated with cancer chemotherapy comprises selecting a patient in need of treatment and/or prevention and/or control of nausea and/or vomiting associated with cancer chemotherapy; and topically applying or instructing to topically apply the pharmaceutical composition as described herein, wherein the topically applying is performed at least once in a day.

In an embodiment, the composition is a liquid formulation and/or a semisolid formulation, wherein the topically applying is done two to six times in a day, once in a day, once in two days, once in three days, once in four days, once in five days, once in six days, once in a week.

In an embodiment, the composition is topically applied once in two days, once in three days, once in four days, once in five days, once in six days, once in a week, or once in ten days.

In an embodiment, topically applying provides a constant rate of delivery of the active components of the transdermal patch over a time period.

In an embodiment, topically applying provides a steady absorption rate of dronabinol over a time period.

In an embodiment, topically applying achieves a constant blood serum level of dronabinol over a time period.

In an embodiment, topically applying achieves a reduced variability in blood serum level of dronabinol over a time period relative to oral administration of a dronabinol over the time period. In an embodiment, topically applying achieves a plasma concentration of dronabinol in a therapeutic range over a period of time.

In an embodiment, as method for the treatment and/or prevention and/or control of nausea and/or vomiting associated with cancer chemotherapy and/or anorexia associated with weight loss in patients with AIDS comprises selecting a patient in need of the treatment and/or prevention and/or control of nausea and/or vomiting associated with cancer chemotherapy and/or anorexia associated with weight loss in patients with AIDS; topically applying or instructing to topically apply the pharmaceutical composition described herein; wherein applying achieves topical delivery of dronabinol for the treatment and/or prevention and/or control of nausea and/or vomiting associated with cancer chemotherapy and/or anorexia associated with weight loss in patients with AIDS.

In an embodiment, the pharmaceutical composition is topically applied once in a day, once in two days, once in three days, once in four days, once in five days, once in six days, once in a week, or once in ten days.

In an embodiment, the pharmaceutical composition is topically applied two to six times in a day, once in a day, once in two days, once in three days, once in four days, once in five days, once in six days, once in a week.

In an embodiment, the pharmaceutical composition is a liquid formulation or a semisolid formulation.

In an embodiment, the pharmaceutical composition is a transdermal delivery system.

In addition to the exemplary aspects and embodiments described above, further aspects and embodiments will become apparent by reference to the drawings and by study of the following descriptions.

The disclosure provides a pharmaceutical composition, comprising: dronabinol in a dosage form for transdermal delivery. The disclosure provides a pharmaceutical composition comprising no additional antiemetic drug. The disclosure provides a pharmaceutical composition wherein dronabinol is in a form selected from the group consisting of co-crystals, amorphous, coated, crystalline, a salt, an isomer, a solid solution, a prodrug, an analog, a derivative, a metabolite, a solution, synthetic, an ethanol solution, sesame oil solution and a naturally derived delta-9-tetrahydrocannabinol. The disclosure provides a pharmaceutical composition wherein dronabinol is in the composition at between about 0.01%-95% w/w or between about 0.01%-95% w/v. The disclosure provides a pharmaceutical composition wherein dronabinol is selected from a group consisting of amorphous dronabinol, crystalline dronabinol, co-crystals of dronabinol, coated dronabinol, and ethanolic solution of dronabinol, sesame oil solution of dronabinol in the range of 0.01%-95% w/w or w/v. The disclosure provides a pharmaceutical composition wherein dronabinol is in a salt form. The disclosure provides a pharmaceutical composition formulated as transdermal liquid formulation, transdermal semisolid formulation and/or transdermal polymer matrix formulation. The disclosure provides a pharmaceutical composition comprising a carrier or an ingredient in effective amount either alone or in combinations thereof selected from the group consisting of solvents, gelling agents, polymers, penetration enhancers, emollients, skin irritation reducing agents, buffering agents, pH stabilizers, solubilizers, suspending agents, dispersing agents, stabilizers, plasticizers, surfactants, antioxidants, and oxidants. The disclosure provides a pharmaceutical composition wherein the carrier or ingredient is in the composition in a range of between about 0.01%-95% w/w or w/v. The disclosure provides a pharmaceutical composition wherein the pharmaceutical composition is formulated as a transdermal patch. The disclosure provides a pharmaceutical composition wherein the transdermal patch is selected from the group consisting of a reservoir patch, a micro-reservoir patch, a matrix patch, a pressure sensitive adhesive patch, and an extended release transdermal film. The disclosure provides a pharmaceutical composition wherein the pharmaceutical composition is formulated as microneedles. The disclosure provides a pharmaceutical composition wherein the microneedles are formulated as a transdermal patch.

The disclosure provides a method for the treatment and/or prevention and/or control of nausea and/or vomiting associated with cancer chemotherapy, comprising: selecting a patient in need of treatment and/or prevention and/or control of nausea and/or vomiting associated with cancer chemotherapy; and topically applying or instructing to topically apply the pharmaceutical composition as disclosed herein, wherein the topically applying is performed at least once in a day.

The disclosure provides a method for the treatment and/or prevention and/or control of nausea and/or vomiting associated with cancer chemotherapy wherein the composition is a liquid formulation and/or a semisolid formulation, wherein the topically applying is done two to six times in a day, once in a day, once in two days, once in three days, once in four days, once in five days, once in six days, once in a week. The disclosure provides a method for the treatment and/or prevention and/or control of nausea and/or vomiting associated with cancer chemotherapy wherein the composition is topically applied once in two days, once in three days, once in four days, once in five days, once in six days, once in a week, or once in ten days. The disclosure provides a method for the treatment and/or prevention and/or control of nausea and/or vomiting associated with cancer chemotherapy wherein said topically applying provides a constant rate of delivery of the active components of the transdermal patch over a time period. The disclosure provides a method for the treatment and/or prevention and/or control of nausea and/or vomiting associated with cancer chemotherapy wherein said topically applying provides a steady absorption rate of dronabinol over a time period. The disclosure provides a method for the treatment and/or prevention and/or control of nausea and/or vomiting associated with cancer chemotherapy wherein said topically applying achieves a constant blood serum level of dronabinol over a time period. The disclosure provides a method for the treatment and/or prevention and/or control of nausea and/or vomiting associated with cancer chemotherapy wherein said topically applying achieves a reduced variability in blood serum level of dronabinol over a time period relative to oral administration of a dronabinol over the time period. The disclosure provides a method for the treatment and/or prevention and/or control of nausea and/or vomiting associated with cancer chemotherapy wherein said topically applying achieves a plasma concentration of dronabinol in a therapeutic range over a period of time.

The disclosure provides a method for the treatment and/or prevention and/or control of nausea and/or vomiting associated with cancer chemotherapy and/or anorexia associated with weight loss in patients with AIDS, comprising: selecting a patient in need of the treatment and/or prevention and/or control of nausea and/or vomiting associated with cancer chemotherapy and/or anorexia associated with weight loss in patients with AIDS; topically applying or instructing to topically apply the pharmaceutical composition as disclosed herein, wherein said applying achieves topical delivery of dronabinol for the treatment and/or prevention and/or control of nausea and/or vomiting associated with cancer chemotherapy and/or anorexia associated with weight loss in patients with AIDS. The disclosure provides a method for the treatment and/or prevention and/or control of nausea and/or vomiting associated with cancer chemotherapy and/or anorexia associated with weight loss in patients with AIDS wherein the pharmaceutical composition is topically applied once in a day, once in two days, once in three days, once in four days, once in five days, once in six days, once in a week, or once in ten days. The disclosure provides a method for the treatment and/or prevention and/or control of nausea and/or vomiting associated with cancer chemotherapy and/or anorexia associated with weight loss in patients with AIDS wherein the pharmaceutical composition is topically applied two to six times in a day, once in a day, once in two days, once in three days, once in four days, once in five days, once in six days, once in a week. The disclosure provides a method for the treatment and/or prevention and/or control of nausea and/or vomiting associated with cancer chemotherapy and/or anorexia associated with weight loss in patients with AIDS wherein the pharmaceutical composition is a liquid formulation or a semisolid formulation. The disclosure provides a method for the treatment and/or prevention and/or control of nausea and/or vomiting associated with cancer chemotherapy and/or anorexia associated with weight loss in patients with AIDS wherein the pharmaceutical composition is a transdermal delivery system. The disclosure provides a method for the treatment of a neurological disease or disorder comprising: a. selecting a patient in need of treatment of a neurological disease or disorder; b. topically applying: i. a pharmaceutical composition in the form of a transdermal patch comprising: dronabinol, wherein the concentration of the dronabinol is from about 0.1% to about 15% w/w; wherein the pharmaceutical composition further comprises: ii. about 1% to about 17% w/w of a solvent; iii. about 1% to about 25% w/w of a penetration enhancer; iv. about 35% to about 80% w/w of a pressure sensitive adhesive such as a silicone pressure sensitive adhesive, polyisobutylene adhesive; v. optionally a polymer and/or a stabilizer such as an antioxidant, wherein the neurological disease or disorder is selected from the group consisting of dementia, amyotrophic lateral sclerosis (ALS), frontotemporal dementia (FTD), ALS with FTD, Alzheimer's disease (AD), Parkinson's disease (PD), Huntington's disease, progressive supranuclear palsy (PSP), brain trauma, spinal cord injury, corticobasal degeneration (CBD), nerve injuries, neuropathies, and TDP43 proteinopathies, chronic traumatic encephalopathy, Perry Syndrome, Dementia with Lewy body in association with Alzheimer's disease, Parkinson's disease with or without dementia, and Limbic-predominant age-related TDP-43 encephalopathy (LATE). The disclosure provides a method for the treatment of a neurological disease or disorder wherein dronabinol is in a form selected from the group consisting of co-crystals, amorphous, coated, crystalline, a salt, an isomer, a solid solution, a prodrug, an analog, a derivative, a metabolite, a solution, synthetic, an ethanol solution, sesame oil solution and a naturally derived delta-9-tetrahydrocannabinol. The disclosure provides a method for the treatment of a neurological disease or disorder wherein dronabinol is selected from a group consisting of amorphous dronabinol, crystalline dronabinol, co-crystals of dronabinol, coated dronabinol, and ethanolic solution, sesame oil solution of dronabinol or/and any vegetable oil of dronabinol in the range of 0.01%-95% w/w or w/v. The disclosure provides a method for the treatment of a neurological disease or disorder wherein dronabinol is in neat dronabinol or ethanolic solution of dronabinol or sesame oil solution of dronabinol. The disclosure provides a method for the treatment of a neurological disease or disorder wherein the pharmaceutical composition in the form of a transdermal patch comprises a carrier or an ingredient in effective amount either alone or in combinations thereof selected from the group consisting of solvents, cosolvents, gelling agents, polymers, penetration enhancers, emollients, skin irritation reducing agents, buffering agents, pH stabilizers, solubilizers, suspending agents, dispersing agents, stabilizers, plasticizers, surfactants, emulsifiers, antioxidants, and oxidants. The disclosure provides a method for the treatment of a neurological disease or disorder wherein the pharmaceutical composition in the form of a transdermal patch is formulated as microneedles. The disclosure provides a method for the treatment of a neurological disease or disorder wherein the topical application of a transdermal patch for the treatment of a neurological disease or disorder is selected from the group consisting of once in a day, once in two days, once in three days, once in four days, once in five days, once in six days, once in a week, once in ten days. The disclosure provides a method for the treatment of a neurological disease or disorder wherein the transdermal patch is in the form of a transdermal matrix patch. The disclosure provides a method for the treatment of a neurological disease or disorder wherein the transdermal patch is in the form of a transdermal reservoir patch wherein reservoir formulation is a liquid formulation or a semisolid formulation and formulation does not contain a pressure sensitive adhesive. The disclosure provides a method for the treatment of a neurological disease or disorder wherein the pharmaceutical composition in the form of a transdermal reservoir formulation and comprises a carrier or an ingredient in effective amount either alone or in combinations thereof selected from the group consisting of solvents, cosolvents, gelling agents, polymers, penetration enhancers, emollients, skin irritation reducing agents, buffering agents, pH stabilizers, solubilizers, suspending agents, dispersing agents, stabilizers, plasticizers, surfactants, antioxidants, and oxidants. The disclosure provides a method for the treatment of a neurological disease or disorder further providing a constant rate of delivery of the active components of the transdermal patch over a time period. The disclosure provides a method for the treatment of a neurological disease or disorder further providing a steady absorption rate of the active components of the transdermal patch over a time period. The disclosure provides a method for the treatment of a neurological disease or disorder further achieving a constant blood serum level of the active components of the transdermal patch over a time period. The disclosure provides a method for the treatment of a neurological disease or disorder further achieving a reduced variability in dosage of the active components of the transdermal patches over a time period. The disclosure provides a method for the treatment of a neurological disease or disorder further providing a plasma concentration of the active components of the transdermal patch in a therapeutic range over a period of time. The disclosure provides a method for the treatment of a neurological disease or disorder wherein the neurological disease or disorder is Dementia. The disclosure provides a method for the treatment of a neurological disease or disorder wherein the neurological disease or disorder is Alzheimer's Disease. The disclosure provides a method for the treatment of a neurological disease or disorder wherein the composition is a liquid formulation and/or a semisolid formulation, wherein the topically applying is done two to six times in a day, once in a day, once in two days, once in three days, once in four days, once in five days, once in six days, once in a week.

The disclosure provides a method for the treatment and/or prevention and/or control of seizure disorder in a patient comprising: a. selecting a patient in need of treatment and/or prevention and/or control of seizure disorder; b. topically applying: i. a pharmaceutical composition in the form of a transdermal patch comprising: dronabinol, wherein the concentration of the dronabinol is from about 0.1% to about 15% w/w; wherein the pharmaceutical composition further comprises: ii. about 1% to about 17% w/w of a solvent; iii. about 1% to about 25% w/w of a penetration enhancer; iv. about 35% to about 80% w/w of a silicone pressure sensitive adhesive; at least one suspending agent comprising silicon dioxide; v. an antioxidant comprising BHT, wherein the transdermal patch provides an average flux of the dronabinol of about 0.43 to about 0.93 g/cm²/hr over at least 6 days. The disclosure provides a method for the treatment and/or prevention and/or control of seizure disorder in a patient wherein the seizure disorder includes complex partial seizures, simple partial seizures, partial seizures with secondary generalization, generalized seizures (including absence, grand mal (tonic clonic), status epilepticus, tonic, atonic, myoclonic), neonatal and infantile spasms, drug-induced seizures, trauma-induced seizures, and febrile seizures, and additional specific epilepsy syndromes such as juvenile myoclonic epilepsy, Lennox-Gastaut, Dravet syndrome, Tuberous Sclerosis Complex (TSC), Treatment-Resistant Epilepsy, Treatment Resistant Pediatric Epilepsy, mesial temporal lobe epilepsy, nocturnal frontal lobe epilepsy, progressive epilepsy with mental retardation, and progressive myoclonic epilepsy, as well as seizures associated with CNS mass lesions. The disclosure provides a method for the treatment and/or prevention and/or control of seizure disorder in a patient wherein the topical application of a transdermal patch for the treatment and/or prevention and/or control of seizure disorder in a patient, wherein the seizure disorder include, for example, complex partial seizures, simple partial seizures, partial seizures with secondary generalization, generalized seizures (including absence, grand mal (tonic clonic), status epilepticus, tonic, atonic, myoclonic), neonatal and infantile spasms, drug-induced seizures, trauma-induced seizures, and febrile seizures, and additional specific epilepsy syndromes such as juvenile myoclonic epilepsy, Lennox-Gastaut, Dravet syndrome, Tuberous Sclerosis Complex (TSC), Treatment-Resistant Epilepsy, Treatment Resistant Pediatric Epilepsy, mesial temporal lobe epilepsy, nocturnal frontal lobe epilepsy, progressive epilepsy with mental retardation, and progressive myoclonic epilepsy, as well as seizures associated with CNS mass lesions is selected from the group consisting of once in a day, once in two days, once in three days, once in four days, once in five days, once in six days, once in a week, once in ten days. The disclosure provides a method for the treatment and/or prevention and/or control of seizure disorder in a patient wherein the transdermal patch is in the form of a transdermal matrix patch. The disclosure provides a method for the treatment and/or prevention and/or control of seizure disorder in a patient further providing a constant rate of delivery of the active components of the transdermal patch over a time period. The disclosure provides a method for the treatment and/or prevention and/or control of seizure disorder in a patient further providing a steady absorption rates of the active components of the transdermal patch over a time period. The disclosure provides a method for the treatment and/or prevention and/or control of seizure disorder in a patient further achieving a constant blood serum levels of the active components of the transdermal patch over a time period. The disclosure provides a method for the treatment and/or prevention and/or control of seizure disorder in a patient further achieving a reduced variability in dosage of the active components of the transdermal patches over a time period. The disclosure provides a method for the treatment and/or prevention and/or control of seizure disorder in a patient further providing a plasma concentration of the active components of the transdermal patch in a therapeutic range over a period of time.

The disclosure provides a method of treating one or more symptoms of a psychological disorder in a subject comprising: a. selecting a patient in need of treating one or more symptoms of a psychological disorder; b. topically applying: i. a pharmaceutical composition in the form of a transdermal patch comprising: dronabinol, wherein the concentration of the dronabinol is from about 0.1% to about 15% w/w; wherein the pharmaceutical composition further comprises: ii. about 1% to about 17% w/w of a solvent; iii. about 1% to about 25% w/w of a penetration enhancer; iv. about 35% to about 80% w/w of a silicone pressure sensitive adhesive; at least one suspending agent comprising silicon dioxide; v. an antioxidant comprising BHT, wherein the transdermal patch provides an average flux of the dronabinol of about 0.43 to about 0.93 μg/cm²/hr over at least 6 days, wherein the subject has symptoms of at least one of agitation, agitation associated with CTE, anxiety, stress, insomnia, and/or depression when assessed by a validated scale or biomarker for identifying symptoms of agitation, agitation associated with CTE, anxiety, stress, insomnia, and/or depression. The disclosure provides a method of treating one or more symptoms of a psychological disorder in a subject wherein the subject has symptoms of two or more of agitation, agitation associated with CTE, anxiety, stress, insomnia, and/or depression. The disclosure provides a method of treating one or more symptoms of a psychological disorder in a subject wherein the transdermal patch is in the form of a transdermal matrix patch. The disclosure provides a method of treating one or more symptoms of a psychological disorder in a subject wherein the method: i. reduces the subject's score on the validated scale for anxiety or reduces the agitation, agitation associated with CTE, anxiety biomarker; ii. reduces the subject's score on the validated scale for stress or reduces the stress biomarker; iii. reduces the subject's score on the validated scale for depression or the depression biomarker; and/or reduces the subject's score on the validated scale for insomnia or reduces an insomnia biomarker.

The disclosure provides a pharmaceutical composition in the form of a transdermal patch comprising: a. dronabinol, wherein the concentration of the dronabinol is from about 0.1% to about 15% w/w; wherein the pharmaceutical composition further comprises: b. about 1% to about 17% w/w of a solvent; c. about 1% to about 25% w/w of a penetration enhancer; d. about 35% to about 80% w/w of a silicone pressure sensitive adhesive; at least one suspending agent comprising silicon dioxide; e. an antioxidant comprising BHT. The disclosure provides a pharmaceutical composition in the form of a transdermal patch wherein the transdermal patch is in the form of a transdermal matrix patch. The disclosure provides a pharmaceutical composition in the form of a transdermal patch wherein the transdermal patch is in the form of a transdermal reservoir patch wherein reservoir formulation is a liquid formulation or a semisolid formulation and formulation does not contain a pressure sensitive adhesive. The disclosure provides a pharmaceutical composition in the form of a transdermal patch wherein the pharmaceutical composition in the form of a transdermal reservoir formulation and comprises a carrier or an ingredient in effective amount either alone or in combinations thereof selected from the group consisting of solvents, cosolvents, gelling agents, polymers, penetration enhancers, emollients, skin irritation reducing agents, buffering agents, pH stabilizers, solubilizers, suspending agents, dispersing agents, stabilizers, plasticizers, surfactants, antioxidants, and oxidants.

Additional embodiments of the present methods and compositions, and the like, will be apparent from the following description, drawings, examples, and claims. As can be appreciated from the foregoing and following description, each and every feature described herein, and each and every combination of two or more of such features, is included within the scope of the present disclosure provided that the features included in such a combination are not mutually inconsistent. In addition, any feature or combination of features may be specifically excluded from any embodiment of the present disclosure. Additional aspects and advantages of the present disclosure are set forth in the following description and claims, particularly when considered in conjunction with the accompanying examples and drawings.

### BRIEF DESCRIPTION OF THE DRAWINGS

FIG. 1 is a graph showing the plasma concentration of a drug as a function of time, in hours, when delivered via the oral route (open circles) or the transdermal route (closed circles).

### DETAILED DESCRIPTION

In transdermal drug delivery, a transdermal patch or transdermal composition is applied topically to the skin surface. Throughout the duration of topical application of a transdermal patch or transdermal composition drug is continuously released and delivered through the intact skin (via transcellular, intercellular and trans-appendageal routes) to achieve systemic effect. Therefore, once applied the transdermal composition or transdermal patch can deliver drug into systemic circulation throughout the day or even for more than one day depending on the duration of its application, which can be up to a week or longer.

Transdermal delivery can reduce the dosing frequency of dronabinol which is currently administered orally 4-6 times a day. Through transdermal delivery, transdermal compositions or transdermal formulations or transdermal patch of dronabinol can be applied topically to skin thereby delivering the drug throughout the duration of topical application. Depending on the requirement, duration of topical application can be once in day, once in two days, once in three days, once in four days, once in five days, once in six days, once in a week. Therefore, transdermal delivery can overcome the multiple dose regimen of oral delivery by reducing the dosing frequency.

Moreover, in transdermal drug delivery drug is delivered slowly and continuously throughout the duration of topical application. Preferably drug is delivered at an average flux of between 50 and 2000 ng/cm²/hr (e.g., between 50 and 100 ng/cm²/hr, between 100 and 250 ng/cm²/hr, between 250 and 500 ng/cm²/hr, between 500 and 750 ng/cm²/hr, between 750 and 1000 ng/cm²/hr, between 1000 and 1250 ng/cm²/hr, between 1250 and 1500 ng/cm²/hr, between 1500 and 1750 ng/cm²/hr, between 1750 and 2000 ng/cm²/hr, between 500 and 1000 ng/cm²/hr, between 1000 and 1500 ng/cm²/hr, or between 1500 and 2000 ng/cm²/hr) over the course of the administration of the drug. Drug administration by transdermal delivery reaches a steady state flux at which point the percent relative standard deviation (% RSD) in flow is preferably less than 50% for the duration of the administration (e.g., less than 50%, less than 45%, less than 40%, less than 35%, less than 30%, less than 25%, less than 20%, less than 15%, less than 10%, or less than 5%). The steady state flow period may last between 2 and 7 days (e.g., between 2 and 3 days, between 3 and 4 days, between 4 and 5 days, between 5 and 6 days, between 6 and 7 days, between 2 and 5 days, between 3 and 6 days, or between 4 and 7 days), and may be reached in less than 36 hours (e.g., less than 32 hours, less than 28 hours, less than 24 hours, less than 20 hours, less than 16 hours, less than 12 hours, less than 8 hours, or less than 4 hours). When drug is delivered in this manner drug plasma concentrations are steadier and/or more prolonged than by multiple oral administrations in a day. Therefore, by transdermal delivery of dronabinol patients can have the therapeutic effect of the drug for extended period of time without drastic changes in drug plasma concentration.

In transdermal delivery drug is delivered into systemic circulation through the skin, it escapes the first pass hepatic metabolism therefore to achieve the desired therapeutic activity less drug is required, resulting into less adverse effects or side effects. Dronabinol has high lipid solubility and after oral administration undergoes hepatic first pass metabolism therefore of the administered dose 10%-20% reaches systemic circulation hence, as compared to oral dose with transdermal delivery small dose of dronabinol can give the desired therapeutic effects and can also overcome the disturbing psychiatric symptoms associated with high dose.

Also, the transdermal delta-9-THC delivery described herein provides almost 10 times more in-vitro permeability through human cadaver skin using FDA IIG database for approved drug products listed ingredients, as compared to previous systems.

For example, according to FDA labels for Marinol (dronabinol capsule) were given to healthy volunteers (n=34; 20-25 years) twice a day under fasted condition, the following pharmacokinetic (PK) parameters were found (see Label).

TABLE 1
Summary of Marinol ® PK parameters

| | Mean (S.D.) PK Parameters Value | | |
|---|---|---|---|
| BID | Cₘₐₓ | Median Tₘₐₓ | AUC (0-12) |
| Dose (mg) | ng/ml | (range) hr | ng * hr/mL |
| 2.5 | 1.32 (0.62) | 1.00 (0.50-4.00) | 2.88 (1.57) |
| 5 | 2.96 (1.81) | 2.50 (0.50-4.00) | 6.16 (1.85) |
| 10 | 7.88 (4.54) | 1.50 (0.50-3.50) | 15.2 (5.52) |

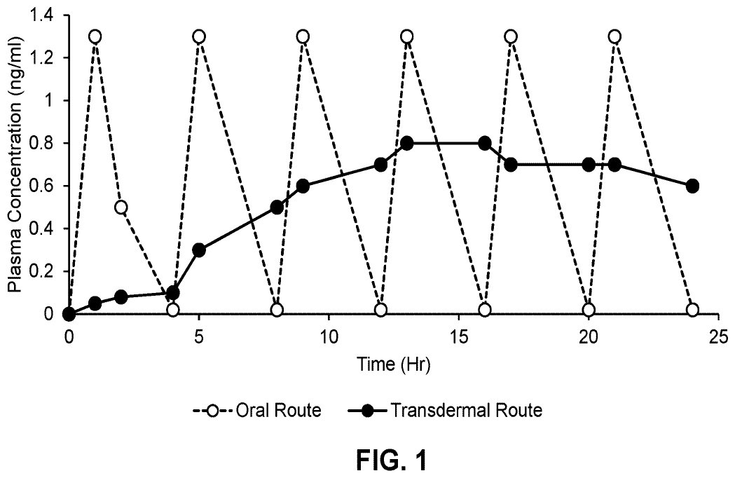

Additionally, according to the PK parameters, the maximum plasma concentration for oral delivery of 5 mg/day by dronabinol capsule is 1.32 ng/mL and for oral delivery of 20 mg/day the maximum plasma concentration is 7.88 ng/mL. The oral delivery PK values demonstrate a peak and valley in plasma concentration over time. In contrast to oral delivery, transdermal drug delivery delivers the drug molecule at predetermined rate and maintains constant average plasma concentration over time (FIG. 1).

The average plasma concentration is calculated in the following manner:

$$Average Plasma Concentration for 5 mg / day = {AUC}_{( 0 - t )} / t = 2.88 / 12 = 0.24 ng / mL 20 mg / day = 15.2 / 12 = 1.27 ng / mL$$

Therefore, transdermal drug delivery systems (TDDS) are calculated to deliver delta-9-THC, at average plasma concentrations of 0.24-1.27 ng/mL.

For the prevention of nausea and vomiting, the oral route is often not the most convenient. Patients are already experiencing nausea and vomiting and if they vomit shortly after the drug administration uncertainty remains whether the dose was absorbed or is vomited. On the other hand, transdermally dronabinol will be delivered through the skin which completely eliminates these kinds of uncertainties.

Moreover, there are various additional side effects, such as Central Nervous System (CNS) side effects, related to oral dronabinol capsule administration. The side effects are dose related and therefore patients receiving the oral dosage require close monitoring so that the dosage can be adjusted and reduced as needed.

Alternatively, the TDDS systems provided herein provide constant drug delivery at a predetermined and defined input rate. Therefore, the currently provided TDDS systems provide a constant average plasma concentration due to constant input rates and do not exhibit PK peaks and valleys in plasma concentration associated with orally delivered delta-9-THC. Accordingly, the currently provided TDDS systems are also not as likely to cause CNS side effects as compared to the orally delivered counterparts. For example, adverse effects of delta-9-THC are not induced with a dosage of 7 mg/m² of delta-9-THC as shown in the following examples. Dosages greater than 7 mg/m² of delta-9-THC had no CINV effect and had more side effects.

Furthermore, oral dosages must be divided by being provided 4 to 6 times per day instead of taking once a day in order to avoid adverse effects. Additionally, oral dosage undergoes first pass metabolism and only 10-20% of the oral dose becomes available in plasma, resulting an inefficient delivery method and a large loss of the active pharmaceutical. Moreover, in order to achieve therapeutic effect from any drug molecule, the drug should have continuous delivery to achieve steady state plasma concentration or a plasma concentration in therapeutic window (between minimum and maximum therapeutic effective concentration). Therefore, the TDDS systems for delta-9-THC provided herein address long felt and unmet needs in the field of cannabidiol based pharmaceuticals.

Also, transdermal delivery is easy, noninvasive and convenient. Administration of transdermal patch or transdermal composition does not require medical supervision as patients can topically apply the transdermal patch or transdermal composition themselves.

With respect to dronabinol it is expected that interpatient variability in pharmacologic response will be less with the transdermal delivery as drug plasma concentration can be controlled by controlling the rate of drug delivery from transdermal composition or transdermal patch. Alpha half-life of dronabinol is about 4 hours, by oral administration it gets rapidly metabolized in the body. In contrast, with transdermal delivery small amount of dronabinol can be delivered for longer duration than oral administration. Transdermal formulations of dronabinol also provide more abuse deterrence than immediate release dosage forms.

Moreover, in case of any adverse effect, side effect or emergency transdermal delivery gives the liberty to terminate the therapy anytime by taking off the transdermal patch or transdermal composition from skin.

As per the above stated reasons for the prevention of nausea and vomiting associated with cancer chemotherapy transdermal delivery can provide patient friendly, simplified and convenient therapeutic regimen over traditional delivery systems. Transdermal delivery can reduce the dosing frequency of dronabinol. Depending on the necessity, dosing frequency can be once in a day, once in two days, once in three days, once in four days, once in five days, once in six days, once in a week.

For the prevention of nausea and vomiting associated with cancer chemotherapy patients are prescribed with a variety of different drugs, some of which are administered multiple times a day. By formulating the antiemetic drug(s) in a transdermal patch or transdermal composition provides a much-simplified dosage regimen to patients who are already weak and tired due to chemotherapy. Depending on the necessity, dosing frequency of the transdermal patch or transdermal composition containing the drug can be once in a day, once in two days, once in three days, once in four days, once in five days, once in six days, once in a week. It would be a great addition to the patient compliance.

Dronabinol is an unstable drug. Stability of dronabinol can be improved by preparation of dronabinol's co crystals, coating the dronabinol, dronabinol's crystals, amorphous form, and/or inert substances, and the like.

Transdermal drug delivery composition, that in one embodiment, are in the form of a transdermal patch or delivery system comprising dronabinol are contemplated. Transdermal delivery can provide drug plasma concentration at predetermined rate for a predetermined period of time with a simplified therapeutic regimen by decreasing dosing frequency. Preferably, without any limitation dronabinol is selected from group such as dronabinol's co crystals, dronabinol's amorphous form, coated dronabinol, dronabinol's crystalline form.

As used herein, “dronabinol” refers to all forms of dronabinol either alone or in combinations thereof, for example, in following forms but not limited to such as free base or salts or isomers or amorphous or crystalline or co-crystals or solid solution or prodrugs or analogs or derivatives or metabolites or coated form or natural extract of delta-9-tetrahydrocannabinol or synthetic delta-9-tetrahydrocannabinol, solution of dronabinol. For example, dronabinol's free form or its salts or its isomers or its amorphous form or its crystalline form or its co crystals or its solid solution or its prodrugs or its analogs or its derivatives or its metabolites or its coated form or natural extract of delta-9-tetrahydrocannabinol or synthetic delta-9-tetrahydrocannabinol, ethanolic solution of dronabinol. Dronabinol may be in the form of a pharmaceutically acceptable salt, such as an acid addition salt or a base salt, or a solvate thereof, including a hydrate thereof. Suitable acid addition salts are formed from acids which form non-toxic salts.
Chemical Name: (6aR-trans)-6a,7, 8, 10a-tetrahydro-6,6,9-trimethyl-3-pentyl-6H-dibenzo dibenzo[b,d]pyran-1-ol
Empirical Formula: C₂₁H₃₀O₂
Molecular weight: 314.47
Structure: Formula 1

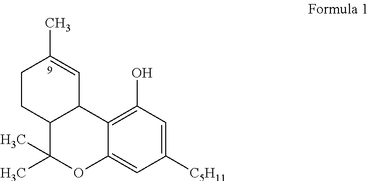

Dronabinol is synthetic delta-9-tetrahydrocannabinol. Delta-9-tetrahydrocannabinol is a natural component, present in Cannabis sativa L

In embodiments of the disclosure, without any limitation, a preferable form of dronabinol is selected from group such as dronabinol co-crystals, dronabinol amorphous form, coated dronabinol, dronabinol crystalline form. Dronabinol's co-crystals, coated dronabinol, dronabinol's crystalline form may be prepared.

Amorphous forms of the drug do not have a definite structure. An amorphous form of the drug has higher solubility as compared to a crystalline form. Different techniques and methods are used to make amorphous form of drugs.

As stated in regulatory classification of pharmaceutical co-crystals guidance for industry “Co-crystals are crystalline materials composed of two or more different molecules, typically drug and co-crystal formers (“coformers”), in the same crystal lattice” (See U.S. Department of Health and Human Services Food and Drug Administration Center for Drug Evaluation and Research (CDER), Regulatory Classification of Pharmaceutical Co-Crystals Guidance for Industry Draft Guidance, Pharmaceutical Quality/CMC Revision 1. August 2016 www.fda.gov/downloads/Drugs/Guidances/UCM516813.pdf, accessed on Jul. 12, 2017). Different methods are available for the preparation of co-crystals. Each drug has distinct chemical structure and physicochemical properties therefore, it is difficult to predict the success rate of co-crystallization reaction. Studies under various experimental conditions are done to determine an approach to form co-crystals of drug (See Nate Schultheiss, Ann Newman. Pharmaceutical Cocrystals and their Physicochemical Properties. Cryst Growth Des. 2009 Jun. 3; 9 (6): 2950-2967).

Coating of the drug can be done with polymer or other excipients. Different techniques are used for the coating of drug. Stability of the drug can also be increased by encapsulation.

As used herein, the term “pharmaceutically acceptable salts” includes acid addition salts or addition salts of free bases. The term “pharmaceutically acceptable salts” of the dronabinol within its scope all the possible isomers and their mixtures, and any pharmaceutically acceptable metabolite, bio-precursor and/or pro-drug, such as, for example, a compound which has a structural formula different from the one of the compounds of the disclosure, and yet is directly or indirectly converted in vivo into a compound of the disclosure, upon administration to a subject, such as a mammal, particularly a human being.

In one embodiment, dronabinol is incorporated into the transdermal system in the form of a pharmaceutically acceptable salt form, either as a single salt, as combinations of salts, or as a combination of the base form and one or more salt forms. Examples of the various forms of dronabinol include but not limited to such as free base, salts, racemic form, isomers, amorphous, crystalline, co-crystals, solid solution, prodrugs, analogs, derivatives, metabolites, solutions, hydrates. Therapeutic agents may be in the form of a pharmaceutically acceptable salt, such as an acid addition salt or a base salt, or a solvate thereof, including a hydrate thereof. Suitable acid addition salts are formed from acids which form non-toxic salts and without any limitation examples are acetate, hydrochloride, hydrobromide, hydroiodide, sulphate, bisulphate, nitrate, phosphate, hydrogen phosphate, sodium phosphate, maleate, fumarate, lactate, tartrate, citrate, gluconate, succinate, saccharate, benzoate, methanesulphonate, ethanesulphonate, benzenesulphonate, p-toluenesulphonate and pamoate salts. Suitable base salts are formed from bases which form non-toxic salts and without any limitation examples are the sodium, potassium, aluminum, calcium, magnesium, zinc and diethanolamine salts.

As used herein, the terms “subject” and “patient” are used interchangeably. As used herein, the term “patient” refers to an animal, preferably a mammal such as a non-primate (e.g., cows, pigs, horses, cats, dogs, rats etc.) and a primate (e.g., monkey and human), and most preferably a human. In some embodiments, the subject is a non-human animal such as a farm animal (e.g., a horse, pig, or cow) or a pet (e.g., a dog or cat). In a specific embodiment, the subject is a human.

As used herein, the term “agent” refers to any molecule, compound, methodology and/or substance for use in the prevention, treatment, management and/or diagnosis of a disease or condition.

As used herein, the term “effective amount” refers to the amount of a therapy that is sufficient to result in the prevention of the development, recurrence, or onset of a disease or condition, and one or more symptoms thereof, to enhance or improve the prophylactic effect(s) of another therapy, reduce the severity, the duration of a disease or condition, ameliorate one or more symptoms of a disease or condition, prevent the advancement of a disease or condition, cause regression of a disease or condition, and/or enhance or improve the therapeutic effect(s) of another therapy.

As used herein, the phrase “pharmaceutically acceptable” means approved by a regulatory agency of the federal or a state government, or listed in the U.S. Pharmacopeia, European Pharmacopeia, or other generally recognized pharmacopeia for use in animals, and more particularly, in humans.

As used herein, the term “therapeutic agent” refers to any molecule, compound, and/or substance that is used for treating and/or managing a disease or disorder.

As used herein, the terms “therapies” and “therapy” can refer to any method(s), composition(s), and/or agent(s) that can be used in the prevention, treatment and/or management of a disease or condition, or one or more symptoms thereof. In certain embodiments, the terms “therapy” and “therapies” refer to small molecule therapy.

The term “derivative” or “derivatized” as used herein includes chemical modification of a compound of the disclosure, or pharmaceutically acceptable salts thereof or mixtures thereof. That is, a “derivative” may be a functional equivalent of a compound of the disclosure, which is capable of inducing the improved pharmacological functional activity in a given subject.

As used herein, the terms “composition” and “formulation” are used interchangeably.

As used herein, the term “topical delivery” means delivery of drug into systemic circulation through the skin.

### Transdermal Compositions

According to certain embodiments, transdermal compositions described herein are for the prevention and/or treatment of nausea and/or vomiting associated with cancer chemotherapy.

According to certain embodiments described herein, the pharmaceutical composition or transdermal formulation contains dronabinol in a form selected from a group such as co-crystals, amorphous form, crystalline form, its coated form, its solution, and its salts which can be anhydrous and/or hydrous alone or in combinations thereof. More preferably the transdermal formulation may include dronabinol selected from following forms: amorphous or co-crystals or crystalline or coated or its ethanolic solution, sesame oil solution, either alone or in combination of thereof.

A transdermal formulation and/or topical formulation may comprise gelling agents and/or thickening and/or suspending agents, alone or in combinations, including but not limited to natural polymers, polysaccharides and its derivatives (such as but not limited to agar, alginic acid and derivatives, cassia tora, collagen, gelatin, gellum gum, guar gum, pectin, potassium, or sodium carageenan, tragacanth, xantham, gum copal, chitosan, resin etc.), semisynthetic polymers and its derivatives such as without any limitation to cellulose and its derivatives (methylcellulose, ethyl cellulose, carboxymethyl cellulose, hydroxylpropyl cellulose, hydroxylpropylmethyl cellulose etc.), synthetic polymers and its derivatives such as without any limitation to carboxyvinyl polymers or carbomers (carbopol 940, carbopol 934, carbopol 971p NF), polyethylene, and its copolymers etc, clays such as but not limited to (silicates, bentonite), silicon dioxide, polyvinyl alcohol, acrylic polymers (eudragit), acrylic acid esters, polyacrylate copolymers, polyacrylamide, polyvinyl pyrrolidone homopolymer and polyvinyl pyrrolidone copolymers such as but not limited to (PVP, Kollidon 30, Poloxamer), isobutylene, ethyl vinyl acetate copolymers, natural rubber, synthetic rubber, pressure sensitive adhesives such as silicone polymers such as but not limited to (bio-psa 4302, bio-psa 4202 etc.), acrylic pressure sensitive adhesives such as but not limited to (DURO-TAK 87-2156, DURO-TAK 387-2287, etc.), polyisobutylene such as but not limited to (polyisobutylene low molecular weight, polyisobutylene medium molecular weight, polyisobutylene 35000 mw, etc.), acrylic copolymers, rubber based adhesives, hot melt adhesives, styrene-butadiene copolymers, bentonite, all water and/or organic solvent swellable polymers, etc. Formulations may also comprise an ointment product such as hydromol described at, for instance, at https://www.hydromol.co.uk/. The mentioned agents are, in an embodiment, in the range of 0.1% 70% w/w or w/v.

One embodiment of the present disclosure can be a transdermal drug delivery system which may include without any limitation a transdermal formulation, a transdermal patch, a topical formulation, microneedles, iontophoresis, or a metered dose transdermal spray. For a review of microneedles see, for example, McConville et al, Medicines, 5:50 (2018), incorporated herein by reference.

Transdermal formulation which includes liquids for example without any limitation like solutions, suspensions, dispersions, emulsion. Transdermal formulation includes semisolids for example without any limitations like gels, ointments, emulsions, creams, suspension, paste, lotion, balm. Liquid formulation and/or gel formulation incorporated in transdermal patch is preferred. Transdermal formulations which include a polymer matrix can be, without any limitation, an adhesive matrix or a non-adhesive matrix.

Without any limitation, transdermal patch may include all transdermal drug delivery systems stated in art preferably but not limited to reservoir patch, matrix patch, bilayer matrix patch, multilayer matrix patch, microreservoir patch, adhesive systems, transdermally applicable tape and other. For details on microreservoir patches see, for example, Stevenson et al, Advanced Drug Delivery Reviews, 64:1590-1602 (2012), incorporated herein by reference. For details on matrix patches see, for example, Nalamachu et al, Journal of Pain Research, 13:2343 (2020), incorporated herein by reference.

In certain embodiments of the present disclosure, a transdermal patch comprises dronabinol contained in a reservoir or a matrix, and an adhesive which allows the transdermal patch to adhere to the skin, allowing the passage of the dronabinol from the transdermal patch through the skin of the patient. The transdermal delivery system can be occlusive, semi-occlusive or non-occlusive, and can be adhesive or non-adhesive.

In some embodiments, the transdermal patches provide for a constant rate of delivery of the active components of the transdermal patch over a predetermined time period. In some embodiments, the predetermined time period is about 24 hours, 48 hours, 72 hours, 96 hours, 120 hours, 144 hours, 7 days, 8 to 13 days, two weeks, or 15 days.

In yet further embodiments, the transdermal patches described herein provide a steady absorption rate of the active components of the transdermal patches by the patient over a predetermined time. In some embodiments, the predetermined time period is 24 hours, 48 hours, 72 hours, 96 hours, 120 hours, 144 hours, 7 days, 8 to 13 days, two weeks, or 15 days.

In yet further embodiments, the transdermal patches described herein provide a constant blood serum level of the active components of the transdermal patches in a patient over a predetermined time. In some embodiments, the predetermined time period is about 24 hours, 48 hours, 72 hours, 96 hours, 120 hours, 144 hours, 7 days, 8 to 13 days, two weeks, or 15 days.

In yet further embodiments, the transdermal patches described herein provide a plasma concentration of the active components of the transdermal patches in a therapeutic range in a patient over a predetermined time. In some embodiments, the predetermined time period is about 24 hours, 48 hours, 72 hours, 96 hours, 120 hours, 144 hours, 7 days, 8 to 13 days, two weeks, or 15 days.

In yet further embodiments, the transdermal patches described herein allow for reduced variability in dosage of active components in a patient over a predetermined time. In some embodiments, the predetermined time period is about 24 hours, 48 hours, 72 hours, 96 hours, 120 hours, 144 hours, 7 days, 8 to 13 days, two weeks, or 15 days.

The topical formulation stated in the art which include, for example without any limitation, semisolids such as ointment, cream, emulsion, micro emulsion, nano emulsion, paste, balms, gels, lotions, mousses. Liquids such as solutions, suspensions, micro suspension, nano suspension, dispersions, nano dispersion etc. Sprays, aerosols, magma, etc. The topical formulation comprising dronabinol can be topically applied to the skin surface for transdermal delivery of dronabinol.

The transdermal formulation and/or topical formulation of some embodiments of the present disclosure may include carriers or ingredients in effective amount either alone or in combinations thereof without any limitation to the following carriers or ingredients such as solvents, gelling agents, polymers, biodegradable polymers, penetration enhancers, emollients, skin irritation reducing agents, buffering agents, pH stabilizers, solubilizers, suspending agents, dispersing agents, stabilizers, plasticizers, tackifiers, surfactants, volatile chemicals, antioxidants, oxidants, chelating agents, complexing agents, diluents, excipients, material to prepare patch, material to prepare a matrix patch or a reservoir patch.

Dronabinol may be dissolved, suspended, dispersed or uniformly mixed in the above stated single carrier, mixture of carriers and combinations of carrier.

The desired optimum transdermal and/or topical formulation of dronabinol may comprise without any limitation to following carriers as stated from Example 1 to Example 11 either alone or in combinations thereof.

### Alzheimer's Disease

Certain embodiments as disclosed herein provide a method of treating Alzheimer's disease, comprising administering a delivery system of the disclosure to deliver dronabinol, CBD, and/or THC, and/or derivatives thereof, to the subject, thereby treating Alzheimer's disease. Alzheimer's disease (AD), commonly known as senile dementia, is a degenerative disease of the central nervous system characterized by progressive cognitive impairment and memory loss. Its clinical manifestations mainly include memory impairment, cognitive impairment, and mental and behavioral disorders. AD is a major public health issue of common concern to countries around the world. With the development of the global aging process, the incidence of AD continues to increase. Deaths caused by AD rank fifth among the major causes of death in my country. At the same time, family caregivers will also face increased emotional distress and negative physical and mental health risks. Alzheimer's disease is the most common cause of senile dementia and is characterized by cognitive deficits related to degeneration of cholinergic neurons.

### Dementia

The present disclosure is directed to compositions and methods of treating dementia in a patient by administering to the patient an effective amount of Dronabinol, CBD, and/or THC, and/or derivatives thereof. According to the disclosure, these methods include the treatment of dementia per se, as well as methods of treating one or more of the symptoms of dementia. As understood by those skilled in the art, the term “dementia” as used herein refers to a condition resulting from damage to the brain. In some embodiments, the dementia results from one or more conditions including, without limitation, Alzheimer's disease, vascular dementia, dementia with lewy bodies, mixed dementia, Parkinson's disease, frontotemporal dementia, Creutzfeldt-Jakob disease, normal pressure hydrocephalus, Huntington's disease, or Wernick Korsakoff syndrome. In some embodiments, the patient has more than one condition.

Also provided are methods for reducing the likelihood that a patient will develop dementia. Such methods comprise administering to the patient an effective amount of Dronabinol, CBD, and/or THC, and/or derivatives thereof.

As those skilled in the art will readily appreciate, the symptoms of dementia are numerous and may vary from patient to patient. The symptoms may be physical, cognitive, psychological, or any combinations thereof. The symptoms may be mild, moderate or severe and/or may worsen over time. Cognitive changes associated with dementia include, without limitation, one or more of memory loss (short and long term), difficulty communicating or finding words, loss of function (difficulty reasoning or problem-solving, handling complex tasks, planning and organizing), difficulty with coordination and motor functions, confusion, inability to recognize sarcasm, repetitive behavior, difficulty coping with change, and disorientation. Psychological changes include, without limitation, one or more of a personality or mood change, apathy, depression, agitation, agitation associated with CTE, anxiety, inappropriate behavior, paranoia, agitation, hallucinations, or a reduction or loss of inhibitions. Physical symptoms include stroke-like symptoms such as muscle weakness or paralysis, drowsiness, becoming slower in physical movements or having difficulties moving, language problems such as not speaking, speaking less than normal, or difficulties in finding the right words, or incontinence.

Common symptoms of dementia include memory loss (short and long term), confusion, difficulty communicating or finding words, inability to recognize sarcasm, mood changes, loss of function, apathy, repetitive behavior, difficulty coping with change, becoming slower in physical movements or having difficulties moving, and any combination of these symptoms. In preferred embodiments, the methods of the disclosure are used to treat memory loss (short and long term) symptoms of dementia. In some embodiments, the methods of the disclosure are used to treat confusion symptoms of dementia. In other embodiments, the methods of the disclosure are used to treat difficulty communicating or finding words of dementia. In other embodiments, the methods of the disclosure are used to treat the inability to recognize sarcasm symptoms of dementia. In other embodiments, the methods of the disclosure are used to treat mood changes symptoms of dementia. In other embodiments, the methods of the disclosure are used to treat loss of function symptoms of dementia. In other embodiments, the methods of the disclosure are used to treat apathy symptoms of dementia. In other embodiments, the methods of the disclosure are used to treat repetitive behavior symptoms of dementia. In other embodiments, the methods of the disclosure are used to treat difficulty coping with change symptoms of dementia. In other embodiments, the methods of the disclosure are used to treat becoming slower in physical movements or having difficulties moving symptoms of dementia.

Other aspects of the disclosure are directed to methods of treating dementia in a patient by administering to the patient an effective amount of Dronabinol, CBD, and/or THC, and/or derivatives thereof.

According to the methods described the patient's dementia progression and/or symptoms are assessed. This assessment can occur prior to initiating treatment with Dronabinol, CBD, and/or THC, and/or derivatives thereof, or any combinations of the foregoing. This assessment can alternatively or also occur during the course of the treatment period with the Dronabinol, CBD, and/or THC, and/or derivatives thereof, or any combinations of the foregoing.

Also included within the scope of the disclosure are kits useful for practicing the described methods. The kits comprise Dronabinol, CBD, and/or THC, and/or derivatives thereof; together with packaging and instructions for using the kit to treat dementia. In some embodiments, the kits comprise Dronabinol, CBD, and/or THC, and/or derivatives thereof; together with packaging and instructions for using the kit to treat dementia. In other embodiments, the kits comprise Dronabinol, CBD, and/or THC, and/or derivatives thereof; together with packaging and instructions for using the kit to treat dementia. In further embodiments, the kits comprise Dronabinol, CBD, and/or THC, and/or derivatives thereof; together with packaging and instructions for using the kit to treat dementia. In yet other embodiments, the kits comprise Dronabinol, CBD, and/or THC, and/or derivatives thereof; together with packaging and instructions for using the kit to treat dementia.

The disclosure is also directed to methods of treating dementia by administering to the patient an effective amount of another pharmaceutical agent for treating dementia or one or more symptoms of dementia. In some embodiments, the other pharmaceutical agent treats Alzheimer's disease, vascular dementia, dementia with lewy bodies, mixed dementia, Parkinson's disease, frontotemporal dementia, Creutzfeldt-Jakob disease, normal pressure hydrocephalus, Huntington's disease, or Wernick Korsakoff syndrome.

### Neurological Disease or Disorder

The disclosure provides a method for the treatment of a neurological disease or disorder comprising: selecting a patient in need of treatment of a neurological disease or disorder; topically applying a pharmaceutical composition comprising Dronabinol, CBD, and/or THC, and/or derivatives thereof, in a dosage form for transdermal delivery wherein the pharmaceutical composition comprises Dronabinol, CBD, and/or THC, wherein the neurological disease or disorder is selected from the group consisting of dementia, amyotrophic lateral sclerosis (ALS), frontotemporal dementia (FTD), ALS with FTD, Alzheimer's disease (AD), Parkinson's disease (PD), Huntington's disease, progressive supranuclear palsy (PSP), brain trauma, spinal cord injury, corticobasal degeneration (CBD), nerve injuries (e.g., brachial plexus injuries), neuropathies (e.g., chemotherapy induced neuropathy), and TDP43 proteinopathies (e.g., chronic traumatic encephalopathy, Perry Syndrome, Dementia with Lewy body in association with Alzheimer's disease, Parkinson's disease with or without dementia, and Limbic-predominant age-related TDP-43 encephalopathy (LATE)). The disclosure provides a method for the treatment of a neurological disease or disorder wherein the topical application of a transdermal patch for the treatment of a neurological disease or disorder is selected from the group consisting of once in a day, once in two days, once in three days, once in four days, once in five days, once in six days, once in a week, once in ten days. The disclosure provides a method for the treatment of a neurological disease or disorder further providing a constant rate of delivery of the active components of the transdermal patch over a time period. The disclosure provides a method for the treatment of a neurological disease or disorder further providing a steady absorption rate of the active components of the transdermal patch over a time period. The disclosure provides a method for the treatment of a neurological disease or disorder further achieving a constant blood serum level of the active components of the transdermal patch over a time period. The disclosure provides a method for the treatment of a neurological disease or disorder further achieving a reduced variability in dosage of the active components of the transdermal patches over a time period. The disclosure provides a method for the treatment of a neurological disease or disorder further providing a plasma concentration of the active components of the transdermal patch in a therapeutic range over a period of time. The disclosure provides a method for the treatment of a neurological disease or disorder wherein the neurological disease or disorder is Dementia. The disclosure provides a method for the treatment of a neurological disease or disorder wherein the neurological disease or disorder is Alzheimer's Disease.

### Seizure

According to certain embodiments, transdermal compositions described herein are for the treatment and/or prevention and/or control of seizure disorder in a patient, wherein the seizure disorder disorders include, for example, complex partial seizures, simple partial seizures, partial seizures with secondary generalization, generalized seizures (including absence, grand mal (tonic clonic), status epilepticus, tonic, atonic, myoclonic), neonatal and infantile spasms, drug-induced seizures, trauma-induced seizures, and febrile seizures, and additional specific epilepsy syndromes such as juvenile myoclonic epilepsy, Lennox-Gastaut, Dravet syndrome, mesial temporal lobe epilepsy, nocturnal frontal lobe epilepsy, progressive epilepsy with mental retardation, and progressive myoclonic epilepsy, as well as seizures associated with CNS mass lesions.

### Epilepsy

Epilepsy is a brain disorder characterized by repeated seizures over time. Types of epilepsy can include, but are not limited to generalized epilepsy, e.g., childhood absence epilepsy, juvenile myoclonic epilepsy, epilepsy with grand-mal seizures on awakening, West syndrome, Lennox-Gastaut syndrome, Dravet syndrome, Tuberous Sclerosis Complex (TSC), Treatment-Resistant Epilepsy, Treatment Resistant Pediatric Epilepsy, partial epilepsy, e.g., temporal lobe epilepsy, frontal lobe epilepsy, benign focal epilepsy of childhood.

Status epilepticus (SE) can include, e.g., convulsive status epilepticus, e.g., early status epilepticus, established status epilepticus, refractory status epilepticus, super-refractory status epilepticus; non-convulsive status epilepticus, e.g., generalized status epilepticus, complex partial status epilepticus; generalized periodic epileptiform discharges; and periodic lateralized epileptiform discharges. Convulsive status epilepticus is characterized by the presence of convulsive status epileptic seizures, and can include early status epilepticus, established status epilepticus, refractory status epilepticus, super-refractory status epilepticus. Early status epilepticus is treated with a first line therapy. Established status epilepticus is characterized by status epileptic seizures which persist despite treatment with a first line therapy, and a second line therapy is administered. Refractory status epilepticus is characterized by status epileptic seizures which persist despite treatment with a first line and a second line therapy, and a general anesthetic is generally administered. Super refractory status epilepticus is characterized by status epileptic seizures which persist despite treatment with a first line therapy, a second line therapy, and a general anesthetic for 24 hours or more.

Non-convulsive status epilepticus can include, e.g., focal non-convulsive status epilepticus, e.g., complex partial non-convulsive status epilepticus, simple partial non-convulsive status epilepticus, subtle non-convulsive status epilepticus; generalized non-convulsive status epilepticus, e.g., late onset absence non-convulsive status epilepticus, atypical absence non-convulsive status epilepticus, or typical absence non-convulsive status epilepticus.

Compositions described herein can also be administered as a prophylactic to a subject having a CNS disorder e.g., a traumatic brain injury, status epilepticus, e.g., convulsive status epilepticus, e.g., early status epilepticus, established status epilepticus, refractory status epilepticus, super-refractory status epilepticus; non-convulsive status epilepticus, e.g., generalized status epilepticus, complex partial status epilepticus; generalized periodic epileptiform discharges; and periodic lateralized epileptiform discharges; prior to the onset of a seizure.

### Seizure

A seizure is the physical findings or changes in behavior that occur after an episode of abnormal electrical activity in the brain. The term “seizure” is often used interchangeably with “convulsion.” Convulsions are when a person's body shakes rapidly and uncontrollably. During convulsions, the person's muscles contract and relax repeatedly.

Based on the type of behavior and brain activity, seizures are divided into two broad categories: generalized and partial (also called local or focal). Classifying the type of seizure helps doctors diagnose whether or not a patient has epilepsy.

Generalized seizures are produced by electrical impulses from throughout the entire brain, whereas partial seizures are produced (at least initially) by electrical impulses in a relatively small part of the brain. The part of the brain generating the seizures is sometimes called the focus.

There are six types of generalized seizures. The most common and dramatic, and therefore the most well-known, is the generalized convulsion, also called the grand-mal seizure. In this type of seizure, the patient loses consciousness and usually collapses. The loss of consciousness is followed by generalized body stiffening (called the “tonic” phase of the seizure) for 30 to 60 seconds, then by violent jerking (the “clonic” phase) for 30 to 60 seconds, after which the patient goes into a deep sleep (the “postictal” or after-seizure phase). During grand-mal seizures, injuries and accidents may occur, such as tongue biting and urinary incontinence.

Absence seizures cause a short loss of consciousness (just a few seconds) with few or no symptoms. The patient, most often a child, typically interrupts an activity and stares blankly. These seizures begin and end abruptly and may occur several times a day. Patients are usually not aware that they are having a seizure, except that they may be aware of “losing time.”

Myoclonic seizures consist of sporadic jerks, usually on both sides of the body. Patients sometimes describe the jerks as brief electrical shocks. When violent, these seizures may result in dropping or involuntarily throwing objects.

Clonic seizures are repetitive, rhythmic jerks that involve both sides of the body at the same time.

Tonic seizures are characterized by stiffening of the muscles.

Atonic seizures consist of a sudden and general loss of muscle tone, particularly in the arms and legs, which often results in a fall.

Seizures described herein can include epileptic seizures; acute repetitive seizures; cluster seizures; continuous seizures; unremitting seizures; prolonged seizures; recurrent seizures; status epilepticus seizures, e.g., refractory convulsive status epilepticus, non-convulsive status epilepticus seizures; refractory seizures; myoclonic seizures; tonic seizures; tonic-clonic seizures; simple partial seizures; complex partial seizures; secondarily generalized seizures; atypical absence seizures; absence seizures; atonic seizures; benign Rolandic seizures; febrile seizures; emotional seizures; focal seizures; gelastic seizures; generalized onset seizures; infantile spasms; Jacksonian seizures; massive bilateral myoclonus seizures; multifocal seizures; neonatal onset seizures; nocturnal seizures; occipital lobe seizures; post traumatic seizures; subtle seizures; Sylvan seizures; visual reflex seizures; or withdrawal seizures.

### Psychological Disorder

The present disclosure has found that the administration of dronabinol can assist with treating or preventing one or more symptoms of a psychological disorder in a subject. The symptoms of psychological disorder may be agitation, agitation associated with CTE, anxiety, stress and/or depression. Additionally, the symptoms may be insomnia. Insomnia is associated with a psychological disorder and the symptoms of agitation, agitation associated with CTE, anxiety, stress and/or depression may lead to symptoms of insomnia and equally the symptoms of insomnia may lead to symptoms of agitation, agitation associated with CTE, anxiety, stress and/or depression.

The present method provides a method of treating or preventing one or more symptoms of a psychological disorder in a subject comprising the step of:
a) administering a dose of between 50 mg/day and 200 mg/day dronabinol for at least one week
   wherein the subject has symptoms of at least one of anxiety, stress and/or depression when assessed by a validated scale or biomarker for identifying symptoms of agitation, agitation associated with CTE, anxiety, stress and/or depression. Preferably, the subject has symptoms of two or more of agitation, agitation associated with CTE, anxiety, stress and/or depression.

The symptoms of a psychological disorder may present as insomnia. Preferably, the method of treating or preventing one or more symptoms of a psychological disorder results in a reduction in insomnia.

Insomnia may present as disturbed sleep, inability to get to sleep, inability to stay asleep and/or sleep that leaves the subject tired upon waking. The insomnia may be assessed using the Insomnia Severity Index (ISI) 7-item self-report questionnaire. Notably, the ISI has been clinically validated to identify and isolate aspects of insomnia severity based on several indicators, such as difficulty falling asleep, staying asleep, waking up too early, degree of impairment with daytime functioning or satisfaction with sleep. More importantly perhaps, this global measure has been employed in number of recent clinical trials to assess the responses in insomnia to a wide variety of treatments.

The present method therefore provides a method of treating or preventing one or more symptoms of a psychological disorder in a subject comprising the step of:
a) administering a dose of between 50 mg/day and 200 mg/day dronabinol for at least one week
   wherein the subject has symptoms of at least one of agitation, agitation associated with CTE, anxiety, stress and/or depression when assessed by a validated scale or biomarker for identifying symptoms of agitation, agitation associated with CTE, anxiety, stress and/or depression and the subject has insomnia as assessed by a validated scale or biomarker for identifying symptoms of insomnia. Preferably, the subject has symptoms of two or more of agitation, agitation associated with CTE, anxiety, stress and/or depression and symptoms of insomnia.

The present method further provides a method of treating or preventing one or more symptoms of a psychological disorder in a subject comprising the step of:
b) administering a dose of between 50 mg/day and 200 mg/day dronabinol for at least one week
   wherein the subject has symptoms of at least one of agitation, agitation associated with CTE, anxiety, stress and/or depression when assessed by a validated scale for identifying symptoms of agitation, agitation associated with CTE, anxiety, stress and/or depression.

The present method further provides a method of treating or preventing one or more symptoms of a psychological disorder in a subject comprising the step of:
a) administering a dose of between 50 mg/day and 200 mg/day dronabinol for at least one week
   wherein the subject has symptoms of at least one of agitation, agitation associated with CTE, anxiety, stress and/or depression when assessed by a validated scale for identifying symptoms of agitation, agitation associated with CTE, anxiety, stress and/or depression and the subject has insomnia as assessed by a validated scale or biomarker for identifying symptoms of insomnia.

In certain embodiments as disclosed herein the validated scale for identifying symptoms of anxiety, stress and/or depression is chosen from the list comprising: Depression Anxiety Stress Scales (DASS-21, DASS-42), Penn State Worry Questionnaire, Kessler's psychological distress scale 10 (K-10), Hospital Anxiety and Depression Scale (HAD), Hospital Anxiety and Depression Scale-Anxiety Subscale (HADS-A), Beck Anxiety Inventory (BAI), Mood and Anxiety Symptom Questionnaire (MASQ), Generalized Anxiety Disorder Scale (GAD-7), Four-Dimensional Symptom Questionnaire (4DSQ), StateTrait Anxiety Inventory (STAI), Liebowitz Social Anxiety Scale (LSAS), Overall Anxiety Severity and Impairment Scale (OASIS), Patient Health Questionnaire-4 (PHQ-4), Social Interaction Anxiety Scale (SIAS), Social Phobia Inventory (SPIN), Social Phobia and Anxiety Inventory (SPAI), Brief Fear of Negative Evaluation Scale (BFNE), Zung Self-Rating Anxiety/Depression Scale (SAS/SDS), Westside Test Anxiety Scale Validation, Revised Children's Anxiety and Depression Scale (ROADS), Spence Children's Anxiety Scale (SCAS), Anxiety Likert or Visual Analog Scale (VAS), Hamilton Anxiety Rating Scale (HAM-A). In one form, the validated scale is DASS (DASS-21 or DASS-42).

The “symptoms of anxiety” include excessive anxiety and worry (apprehensive expectation) about events or activities (such as social events; work or school performance). The anxiety may be associated with restlessness or feeling keyed up or on edge; being easily fatigued; difficulty concentrating or mind going blank; irritability; muscle tension and/or sleep disturbance (difficulty falling or staying asleep; or restless unsatisfying sleep). The term preferably covers undiagnosed or common anxiety, that is anxiety not diagnosed as an anxiety disorder by a qualified health care provider or physician. However, the term may also include diagnosed disorders such as generalized anxiety disorder, agoraphobia, social anxiety disorder, separation anxiety disorder, selective mutism, specific phobia, panic disorder, agoraphobia, substance/medication-induced anxiety disorder, and anxiety disorder due to another medical condition. The anxiety may be an anxiety disorder as defined in the Diagnostic and Statistical Manual of Mental Disorders (DSM-5).

The “symptoms of depression” include feeling sad or having a depressed mood; loss of interest or pleasure in activities once enjoyed; changes in appetite; weight loss or gain unrelated to dieting; trouble sleeping or sleeping too much; loss of energy or increased fatigue; increase in purposeless physical activity (e.g., inability to sit still, pacing, handwringing) or slowed movements or speech; feeling worthless or guilty; difficulty thinking, concentrating or making decisions; thoughts of death or suicide. The term preferably covers undiagnosed depression. However, the term may also include depression diagnosed by a qualified health care provider or physician. The depression may be Major Depressive Disorder as defined in the Diagnostic and Statistical Manual of Mental Disorders (DSM-5).

The “symptoms of stress” include feeling overwhelmed; inability to concentrate; poor judgment; seeing only the negative; anxious or racing thoughts; constant worrying; agitation; moodiness, irritability, or anger; feeling overwhelmed; loneliness and isolation; aches and pains; diarrhea or constipation; nausea, dizziness; chest pain, rapid heart rate; loss of sex drive; frequent colds or flu; eating more or less; sleeping too much or too little; withdrawing from others; procrastinating or neglecting responsibilities; nervous habits (e.g. nail biting, pacing); using alcohol, cigarettes, or drugs to relax. The term preferably covers stress which has not been diagnosed as a major heath issue by a qualified health care provider or physician. However, the term may also include stress which has been diagnosed as a major heath issue by a qualified health care provider or physician, for example a Trauma- and Stressor-Related Disorder as defined in the DSM-5.

The “symptoms of insomnia” include finding it hard to go to sleep; frequent waking during the night; problems returning to sleep after awakenings; waking up early and being unable to go back to sleep; still feeling tired after waking up; finding it hard to nap during the day even though the subject is tired; feeling tired and irritable during the day. The term preferably covers insomnia which has not been diagnosed as a major heath issue by a qualified health care provider or physician. However, the term may also include insomnia which has been diagnosed as a major heath issue by a qualified health care provider or physician, for example insomnia or an insomnia disorder as defined in the International Classification of Sleep Disorders, 3rd edition (ICSD-3) and the Diagnostic and Statistical Manual for Mental Disorders, 5th edition (DSM-5).

Preferably the subject has symptoms of agitation, agitation associated with CTE, anxiety, stress and/or depression secondary to a medical condition, more preferably a chronic medical condition. The medical condition may be one of the following: cancer, cancer treatment, chronic diseases such as heart and lung disease or diabetes, migraine, insomnia or sleep disorder, chronic pain; gastrointestinal disorder; neurological disorder, osteoarthritis or inflammatory arthritis. The medical condition may be mental illness that is not a mental disorder corresponding to the DSM-5 criteria for: depressive disorders, agitation, agitation associated with CTE, anxiety disorder trauma- and stressor-related disorders, obsessive-compulsive and related disorders.

In certain embodiments the administration of dronabinol is expected to treat or prevent one or more symptoms of a psychological disorder in a subject wherein the subject has symptoms of anxiety, stress or depression or insomnia. For example, the dronabinol may reduce the incidence and/or severity of episodes of agitation, agitation associated with CTE, anxiety, stress or depression or insomnia. Therapeutic effects of the compositions and methods of the present disclosure include, but are not limited to, reduction in frequency and/or severity of episodes of agitation, agitation associated with CTE, anxiety, stress or depression or insomnia.

Compositions and methods of the present disclosure may treat the symptoms of anxiety, stress or depression or insomnia by reducing the incidence and/or severity of one or more of the symptoms of agitation, agitation associated with CTE, anxiety, stress or depression or insomnia, such as those listed above in relation to “symptoms of anxiety”, “symptoms of depression”, “symptoms of stress” and/or “symptoms of insomnia”. Compositions and methods of the present disclosure may prevent the onset of one or more of the symptoms of agitation, agitation associated with CTE, anxiety, stress or depression or insomnia, such as those listed above in relation to “symptoms of anxiety”, “symptoms of depression”, “symptoms of stress” and/or “symptoms of insomnia”.

In certain embodiments, the administration of dronabinol is expected to improve the symptoms of agitation, agitation associated with CTE, anxiety, stress or depression or insomnia. By “improve”, it is meant that there is a reduction in frequency and/or severity of episodes of agitation, agitation associated with CTE, anxiety, stress or depression or insomnia.

Preferably, the treatment reduces one or more symptoms of a psychological disorder in a subject wherein the subject has symptoms when assessed using a validated scale by a drop from a higher score or category to a score or lower category for at least one of agitation, agitation associated with CTE, anxiety, stress or depression or insomnia. In this respect, it does not matter what the original score or category was in the validated scale, merely that the treatment has reduced that score or category to a lower score or category. More preferably, the treatment reduces the symptoms of agitation, agitation associated with CTE, anxiety, stress or depression by a drop from a higher score or category to a lower score or category for at least two of anxiety, stress or depression. For example, if DASS is being used, the treatment may reduce the anxiety score from a category of “very severe” to “severe”, or reduce the depression score from “moderate” to “mild”. The treatment further preferably reduces the symptoms of insomnia by a drop from a higher score or category to a lower score or category. For example, if 151 is being used, the treatment may reduce the insomnia score from a category of “severe” to “moderate”.

Preferably, the subject being treated scores at least:
a) 8 for anxiety (mild-moderate);
b) 15 for stress (mild-moderate); and/or
c) 10 for depression (mild-moderate)
   on at least one of the categories of anxiety, stress or depression when assessed by the Depression Anxiety Stress Scale (DASS-21 or DASS-42); and the treatment reduces the subject's DASS score on at least one of the categories of anxiety, stress or depression.

Preferably, the subject being treated scores at least 8 for insomnia when assessed by the Insomnia Severity Index (ISI) 7-item self-report questionnaire; and the treatment reduces the subject's ISI score.

As the symptoms of a psychological disorder include agitation, agitation associated with CTE, anxiety, stress or depression, and can also include insomnia, it is preferred that a reduction in scores for one set of symptoms is associated with a reduction in scores for a second set of symptoms. For example, without being held to any theory, as the symptoms of anxiety, stress and/or depression may lead to symptoms of insomnia and equally the symptoms of insomnia may lead to symptoms of anxiety, stress and/or depression, it is preferred that reduction in one or more of the symptoms results in a reduction in at least one other symptom. For example, reducing the symptoms of agitation, agitation associated with CTE, anxiety may assist in reducing the symptoms of insomnia, or reducing the symptoms of insomnia may assist in reducing the symptoms of stress.

The reduction in the symptoms of a psychological disorder of agitation, agitation associated with CTE, anxiety, stress or depression or insomnia may not occur immediately after administration of the first dose of dronabinol. The reduction in the symptoms of a psychological disorder may take days, weeks or months to be detectable by use of a validated scale. In one embodiment, the reduction in the symptoms of agitation, agitation associated with CTE, anxiety, stress or depression is detectable by a drop from a higher score or category to a score or lower category for at least one of anxiety, stress or depression over a time period of one week, two weeks, three weeks or one month.

Preferably, the reduction in the symptoms of agitation, agitation associated with CTE, anxiety, stress or depression is detectable by a drop from a higher score or category to a score or lower category for at least one of agitation, agitation associated with CTE, anxiety, stress or depression or insomnia is maintained for at least as long as the dronabinol is being administered. For example, the reduction in the symptoms of agitation, agitation associated with CTE, anxiety, stress or depression or insomnia may be maintained for at least 1 month, 2 months, 6 months, 12 months, 2 years or indefinitely whilst the dronabinol is being administered. In addition, the reduction in the symptoms of agitation, agitation associated with CTE, anxiety, stress or depression or insomnia may be maintained for at least 1 month, 2 months, 6 months, 12 months, 2 years or indefinitely after administration of the last dose of dronabinol.

The composition may be administered daily for at least one week, two weeks, three weeks, one month. The daily administration may be continued for two months, three months, four months, five months, six months, seven months, eight months, nine months, ten months, eleven months or twelve months. The daily delivery may be administered for one week, two weeks, three weeks, one month or a few months, then there may be a break in delivery for several months, until symptoms of a psychological disorder recur and treatment is resumed.

### EXAMPLES

### Example 1

A transdermal formulation and/or topical formulation comprises solvents, alone or in combinations thereof, including but not limited to alcohol C1-C20 such as but not limited to (methanol, ethanol, isopropyl alcohol, butanol, propanol etc.), polyhydric alcohols, glycols such as but not limited to (propylene glycol, polyethylene glycol, dipropylene glycol, hexylene glycol, butylene glycol, glycerine etc.), derivative of glycols, pyrrolidone such as but not limited to (N methyl 2-pyrrolidone, 2-pyrrolidone etc.), sulfoxides such as but not limited to (dimethyl sulfoxide, decymethylsulfoxide etc), dimethylisosorbide, mineral oils, vegetable oils, olive oil, castor oil, sesame seed oil, soybean oil, etc., water, polar solvents, semi polar solvents, non-polar solvents, volatile chemicals which can be used to make matrix patch such as but not limited to (ethanol, propanol, ethyl acetate, acetone, methanol, dichloromethane, chloroform, toluene, IPA), acids such as but not limited to acetic acid, lactic acid, levulinic acid, fatty acids, bases and others. Such process solvents as Heptane, Tetrahydrofuran.

The mentioned solvents are, in an embodiment, in the range of 0.01%-95% w/w or w/v.

### Example 2

A transdermal formulation and/or topical formulation comprises gelling agents and/or thickening and/or suspending agents, alone or in combinations, including but not limited to natural polymers, polysaccharides and its derivatives such as but not limited to (agar, alginic acid and derivatives, cassia tora, collagen, gelatin, gellum gum, guar gum, pectin, potassium, or sodium carageenan, tragacanth, xantham, gum copal, chitosan, resin etc.), semisynthetic polymers and its derivatives such as without any limitation to cellulose and its derivatives (methylcellulose, ethyl cellulose, carboxymethyl cellulose, hydroxylpropyl cellulose, hydroxylpropylmethyl cellulose etc.), synthetic polymers and its derivatives such as without any limitation to carboxyvinyl polymers or carbomers (carbopol 940, carbopol 934, carbopol 971p NF), polyethylene, and its copolymers etc, clays such as but not limited to (silicates, bentonite), silicon dioxide, polyvinyl alcohol, acrylic polymers (eudragit), acrylic acid esters, polyacrylate copolymers, polyacrylamide, polyvinyl pyrrolidone homopolymer and polyvinyl pyrrolidone copolymers such as but not limited to (PVP, Kollidon 30, Poloxamer), isobutylene, ethyl vinyl acetate copolymers, natural rubber, synthetic rubber, pressure sensitive adhesives such as silicone polymers such as but not limited to (bio-psa 4302, bio-psa 4202, bio-psa 4501, bio-psa 4502, etc.), acrylic pressure sensitive adhesives such as but not limited to (DURO-TAK 87-2156, DURO-TAK 387-2287, durotak 9301, durotak 4098, etc.), polyisobutylene such as but not limited to (polyisobutylene low molecular weight, polyisobutylene medium molecular weight, polyisobutylene 35000 mw, etc, Durotak 6908), acrylic copolymers, rubber based adhesives, hot melt adhesives, styrene-butadiene copolymers, bentonite, all water and/or organic solvent swellable polymers, etc. The mentioned agents are, in an embodiment, in the range of 0.1% 70% w/w or w/v.

### Example 3

The transdermal formulation and/or topical formulation of the disclosure may comprise permeation enhancers known to those skilled in the art either alone or in combination thereof without any limitation to the following, such as sulfoxides, and similar chemicals such as but not limited to (dimethylsulfoxide, dimethylacetamide, dimethylformamide, decymethylsulfoxide, dimethylisosorbide etc), azone, pyrrolidones such as but not limited to (N-methyl-2-pyrrolidone, 2-pyrrolidon etc.), esters, fatty acid esters such as but not limited to (propylene glycol monolaurate, butyl ethanoate, ethyl ethanoate, isopropyl myristate, isopropyl palmitate, methyl ethanoate, decyl oleate, glycerol monooleate, glycerol monolaurate, lauryl laurate etc.), fatty acids such as but not limited to (capric acid, caprylic acid, lauric acid, oleic acid, myristic acid, linoleic acid, stearic acid, palmitic acid etc.), alcohols, fatty alcohols and glycols such as but not limited to (oleyl alcohol, nathanol, dodecanol, propylene glycol, glycerol etc.), ethers alcohol such as but not limited to (diethylene glycol monoethyl ether), urea, triglycerides such as but not limited to triacetin, polyoxyethylene fatty alcohol ethers, polyoxyethylene fatty acid esters, esters of fatty alcohols, essential oils, surfactant type enhancers such as but not limited to (brij, sodium lauryl sulfate, tween, polysorbate), terpene, terpenoids, Phenol ethers enhancers such as vanillyl butyl ether (Hot ACT VBE)_and all penetration or permeation enhancers referred in the book. “Percutaneous Penetration Enhancers” (Eric W. Smith, Howard I. Maibach, 2005. November, CRC press). The mentioned agents are, in an embodiment, in the range of 0.01%-95% w/w or w/v.

### Example 4

A transdermal formulation and/or topical formulation comprises plasticizers alone or in combination thereof, including without any limitation glycerol and its esters, phosphate esters, glycol derivatives, sugar alcohols, sebacic acid esters, citric acid esters, tartaric acid esters, adipate, phthalic acid esters, triacetin, oleic acid esters and all the plasticizers which can be used in transdermal drug delivery system referred in the book “Handbook of Plasticizers” (George Wypych, 2004, Chem Tec Publishing). The mentioned agents are, in an embodiment, in the range of 0.01%-95% w/w or w/v.

### Example 5

A transdermal formulation and/or topical formulation comprises emollients, humectants, and/or skin irritation reducing agents either alone or in combinations thereof without any limitation to following like petrolatum, lanolin, mineral oil, dimethicone, zinc oxide, glycerin, propylene glycol and others. The mentioned agents are, in an embodiment, in the range of 0.01%-95% w/w or w/v.

### Example 6

A transdermal formulation and/or topical formulation comprises solubilizers, surfactants, emulsifiers, dispersing agents alone or in combination thereof without any limitation to following: surfactants, including anionic, cationic, nonionic and amphoteric surfactants, such as the sorbitan oleates under the trade name SPAN such as but not limited to SPAN 80, SPAN 20 etc. and the polysorbates such as but not limited to Polysorbate 20, Polysorbate 40, Polysorbate 60, Polysorbate 80 etc., propylene glycol monocaprylate type I, propylene glycol monocaprylate type II, propylene glycol dicaprylate, medium chain triglycerides, propylene glycol monolaurate type II, linoleoyl polyoxyl-6 glycerides, oleoyl-polyoxyl-6-glycerides, lauroyl polyoxyl-6-gylcerides, polyglyceryl-3-dioleate, diethylene glycol monoethyl ether, propylene glycol monolaurate type I, polyglyceryl-3-dioleate, caprylocaproyl polyoxyl-8 glycerides etc, cyclodextrins and others. The mentioned agents are, in an embodiment, in the range of 0.01% 95% w/w or w/v.

### Example 7

Different techniques and ingredients can be used to increase the stability and/or solubility of dronabinol in the formulation such as without any limitation coating, encapsulation, microencapsulation, nanoencapsulation, lyophilization, chelating agents, complexing agents, etc.

### Example 8

A transdermal formulation and/or topical formulation comprises auxiliary pH buffering agents and pH stabilizers and similar compounds known to those skilled in the art to maintain the appropriate pH of formulation preferably in the range of 4.0-8.0 either alone or in combination thereof without any limitation to following such as phosphate buffer, acetate buffer, citrate buffer, etc., acids such as but not limited to (carboxylic acids, inorganic acids, sulfonic acids, vinylogous carboxylic acids, fatty acids, and others), base such as but not limited to (sodium hydroxide, potassium hydroxide, ammonium hydroxide, triethylamine, sodium carbonate, sodium bicarbonate) etc. The mentioned agents are, in an embodiment, in the range of 0.01%-30% w/w or w/v.

### Example 9

A transdermal formulation and/or topical formulation comprises antioxidants, such as but not limited to, sodium metabisulfite, citric acid, ascorbic acid, BHA, and BHT, alpha thioglycerol, histidine, alpha tocopherol, oxidizing agents, stabilizers, discoloring agents, preservatives and similar compounds or chemicals to provide a stable formulation. The mentioned agents are, in an embodiment, in the range of 0.01%-50% w/w or w/v.

### Example 10

A transdermal formulation and/or topical formulation with dronabinol formulated in ointment and/or cream base, gels, lotions, and other topical formulations.

### Example 11

Materials to make the transdermal delivery system of the disclosure in patch form known to those skilled in the art, for example, such as but not limited to reservoir patch, matrix patch, drug in adhesives, transdermal films and may include, such as but are not limited to polymers, copolymers, derivatives, backing film, release membranes, release liners, etc. either alone or in combinations thereof. Pressure sensitive adhesives (such as but not limited to silicone polymers, rubber based adhesives, acrylic polymers, acrylic copolymers, polyisobutylene, acrylic acid-isooctyl acrylate copolymer, hot melt adhesives, polybutylene etc.), backing film (such as but not limited to ethylene vinyl acetate copolymers, vinyl acetate resins, polyurethane, polyvinyl chloride, metal foils, polyester, aluminized films, polyethylene, etc.), release membrane (such as but not limited to microporous polyethylene membrane, microporous polypropylene membrane, rate controlling ethylene vinyl acetate copolymer membrane etc.), release liners (such as but not limited to siliconized polyester films, fluoropolymer coated polyester film, polyester film, siliconized polyethylene terephthalate film, etc.), tapes, etc.

The transdermal formulation and/or topical formulation and/or transdermal delivery system of the disclosure may deliver at least therapeutic effective dose of dronabinol. Therapeutic effective dronabinol dose refers to the therapeutic concentration of dronabinol in human plasma required for treating and/or preventing nausea and/or vomiting associated with chemotherapy. Furthermore, the precise therapeutic effective dose of dronabinol in the transdermal formulation or topical formulation or transdermal delivery system can be determined by those skilled in the art based on factors such as but not limited to the patient's condition etc. The transdermal formulation or topical formulation or transdermal delivery system will be available in different dosage strengths and patch sizes in order to achieve optimum therapeutic outcome based on patient's requirement.

In another embodiment, the transdermal formulation and/or topical formulation and/or transdermal delivery system of the disclosure may deliver at least therapeutic effective dose of dronabinol. Therapeutic effective dronabinol dose refers to the therapeutic concentration of dronabinol in human plasma required for treating as stated in MARINOL label “anorexia associated with weight loss in patients with AIDS” (See Label). Furthermore, the precise therapeutic effective dose of dronabinol in the transdermal formulation or topical formulation or transdermal delivery system can be determined by those skilled in the art based on factors such as but not limited to the patient's condition etc. The transdermal formulation or topical formulation or transdermal delivery system will be available in different dosage strengths and patch sizes in order to achieve optimum therapeutic outcome based on patient's requirement.

The transdermal formulation or transdermal patch of dronabinol preferably but not limited to can be applied to the skin surface in any of the following dosage regimens such as once in a day, once in two days, once in three days, once in four days, once in five days, once in six days, once in a week, once in a 8 to about 13 days, once in two weeks, once in 15 days.

### Example 12

Synthetic delta-9-THC formulations for transdermal delivery (Formulation Nos. 001, 002, 006, 007, 008 and 009) were prepared by mixing ingredients as shown in Table 2:

TABLE 2
Transdermal Synthetic delta-9-THC formulations

| | 001 | 002 | 006 | 007 | 008 | 009 |
|---|---|---|---|---|---|---|
| | (% | (% | (% | (% | (% | (% |
| Ingredients | W/V) | W/V) | W/V) | W/V) | W/V) | W/V) |
| TP | 25 | | | | | |
| PG | 25 | 35 | 89.2 | 88.4 | 33 | 32.7 |
| Water | 20 | 20 | 9.9 | 9.9 | 33 | 32.7 |
| Ethanol | 29.5 | 44.5 | | | 33 | 32.7 |
| THC | 0.5 | 0.5 | 0.9 | 1.8 | 0.95 | 1.9 |

Abbreviations:
TP = Transcutol P;
PG = propylene glycol;
THC = tetrahydrocannabinol.

All of the components from Table 2, with the exception of the THC, were mixed together with stirring for 18 hours. Next, the THC in ethanol was added into the excipient mixture to prepare the final transdermal formulations.

The prepared transdermal formulations were then subjected to a flux measurement test as follows. Human cadaver skin, stored at −80° C., was thawed at room temperature in phosphate buffered saline (PBS), and visually inspected for defects before using in the study. Transdermal flux was then measured using standard Franz diffusion cells composed of a cylindrical donor compartment and a separate water jacketed cylindrical receptor compartment with the volume of 13 mL. The human cadaver skin was clamped between the two compartments with the dermis side facing toward the receptor compartment. The donor compartment was filled with the transdermal THC formulations prepared as described above. The receptor compartment was filled with receptor medium, held at constant temperature, and constantly stirred to collect the THC as it diffuses through the skin and into receptor compartment. It is important to confirm that the receptor fluid is always in contact with the skin. The receptor compartment was emptied at 24 hr intervals for assay of delta-9-THC and replaced with fresh receptor solution. In order to maintain the sink condition in receptor compartment, it is important keep the delta-9-THC concentration in receptor compartment less than 10% of its solubility. The experimental conditions are provided in Table 3:

TABLE 3
Experimental Condition for In-vitro Permeability testing

| | De-ionized water + 0.5% |
|---|---|
| | Brij-O(20) + 0.01% |
| Receiving Media | Sodium Azide |
| Receiving Media Volume (mL) | 13 |
| Sample Volume (mL) | 13 |
| Sampling Interval (hr) | 24 |
| Franz-cell diffusion area (sqcm) | 1.76 |
| Membrane Type | Human Cadaver Skin |

Flux of THC through the human cadaver skin was measured for a minimum period of 96 Hrs (4 days) and results of the flux measurement are provided in Table 4.

TABLE 4
THC Flux Results

| | 001 | 002 | 006 | 007 | 008 | 009 |
|---|---|---|---|---|---|---|
| Total | 98700 | 154400 | 64200 | 122700 | 90900 | 102800 |
| Amount | | | | | | |
| of THC | | | | | | |
| Permeated | | | | | | |
| at 96 hrs | | | | | | |
| (ng/cm²) | | | | | | |
| Flux (ng/ | 1000 | 1600 | 700 | 1300 | 950 | 1070 |
| cm²/hr) | | | | | | |

### Example 13

Additional synthetic delta-9-THC formulations for transdermal delivery (Formulation Nos. 010 through 018) were prepared by mixing ingredients as shown in Table 5:

TABLE 5
Transdermal Synthetic delta-9-THC formulation nos. 010 to 018

| | 010 | 011 | 012 | 013 | 014 | 015 | 016 | 017 | 018 |
|---|---|---|---|---|---|---|---|---|---|
| Ingredients | (% W/V) | (% W/V) | (% W/V) | (% W/V) | (% W/V) | (% W/V) | (% W/V) | (% W/V) | (% W/V) |
| THC | 1.3 | 2.0 | 2.8 | 1.3 | 2.0 | 2.8 | 2.0 | 2.8 | 2.0 |
| Ethanol | 27.3 | 42.4 | 58.7 | 27.3 | 42.4 | 58.7 | 42.4 | 58.7 | 42.4 |
| PG | 64.3 | 50 | 34.6 | 71.4 | 55.6 | 38.5 | — | — | — |
| Water | 7.1 | 5.6 | 3.9 | — | — | — | — | — | — |
| Dimethyl | — | — | — | — | — | — | 55.6 | 38.5 | 55.6 |
| Sulfoxide | | | | | | | | | |
| (DMSO) | | | | | | | | | |

Abbreviations:
THC = tetrahydrocannabinol;
PG = propylene glycol.

Synthetic delta-9-THC formulations for transdermal delivery (010-018) were prepared by the same procedure described in Example 12. Flux measurement was also performed as described in Example 12. The experimental conditions are the same as provided in Table 3 of Example 12.

Flux of THC through the human cadaver skin was measured for a minimum period of 96 Hrs (4 days) and results of the flux measurement experiments are provided in Table 6.

TABLE 6
THC Flux Results

| | Formulation No. | | | | | | | | |
|---|---|---|---|---|---|---|---|---|---|
| | 010 | 011 | 012 | 013 | 014 | 015 | 016 | 017 | 018 |
| Total | 75600 | 94900 | 102800 | 54000 | 145500 | 204600 | 35200 | 35800 | 53400 |
| Amount of | (31%) | (20%) | (52%) | (33%) | (63%) | (43%) | (22%) | (1%) | (6%) |
| THC | | | | | | | | | |
| Permeated | | | | | | | | | |
| at 96 hrs | | | | | | | | | |
| (ng/cm²) | | | | | | | | | |
| Flux | 800 | 1000 | 1070 | 600 | 1500 | 2100 | 400 | 400 | 600 |
| (ng/cm²/hr) | | | | | | | | | |

### Example 14

Additional synthetic delta-9-THC formulations for transdermal delivery (Formulation Nos. 019 to 027) were prepared by mixing ingredients as shown in Table 7.

TABLE 7
Transdermal Synthetic delta-9-THC formulation nos. 019-027

| | 019 | 020 | 021 | 022 | 023 | 024 | 025 | 026 | 027 |
|---|---|---|---|---|---|---|---|---|---|
| | (% | (% | (% | (% | (% | (% | (% | (% | (% |
| Ingredients | W/V) | W/V) | W/V) | W/V) | W/V) | W/V) | W/V) | W/V) | W/V) |
| THC | 2.0 | 2.0 | 2.0 | 2.0 | 2.0 | 2.0 | 2.0 | 2.0 | 2.0 |
| Ethanol | 42.4 | 42.4 | 42.4 | 42.4 | 42.4 | 42.4 | 42.4 | 42.4 | 42.4 |
| PG | 44.4 | 44.4 | 44.4 | 44.4 | 44.4 | 44.4 | 44.4 | 44.4 | 44.4 |
| NMP | 11.1 | | | | | | | | |
| Lauric acid | | 11.1 | | | | | | | |
| Oleic Acid | | | 11.1 | | | | | | |
| Lauryl Lactate | | | | 11.1 | | | | | |
| Oleyl Alcohol | | | | | 11.1 | | | | |
| GMO | | | | | | 11.1 | | | |
| Lactic Acid | | | | | | | 11.1 | | |
| HPC | | | | | | | | 11.1 | |
| (Klucel ®) | | | | | | | | | |

Abbreviations:
THC = tetrahydrocannabinol;
PG = propylene glycol;
NMP = n-methyl pyrrolidone;
GMO = glycerol monooleate;
HPC = hydroxypropyl cellulose.

Abbreviations: THC=tetrahydrocannabinol; PG=propylene glycol; NMP=n-methyl pyrrolidone; GMO=glycerol monooleate; HPC=hydroxypropyl cellulose.

Synthetic delta-9-THC formulations for transdermal delivery (019-027) were prepared by the same procedure described in Example 12. Flux measurement was also performed as described in Example 12. The experimental conditions are the same as provided in Table 3 of Example 12.

Flux of THC through the human cadaver skin was measured for a minimum period of 72 hours (3 days) and results of the flux measurement experiments are provided in Table 8.

TABLE 8
THC Flux Results

| | Formulation No. | | | | | | | | |
|---|---|---|---|---|---|---|---|---|---|
| | 019 | 020 | 021 | 022 | 023 | 024 | 025 | 026 | 027 |
| Total | 56300 | 29600 | 20500 | 5100 | 5100 | 26100 | 36400 | 31300 | 31300 |
| Amount | (10%) | (82%) | (14%) | (29%) | (22%) | (34%) | (92%) | (19%) | (16%) |
| of THC | | | | | | | | | |
| Permeated | | | | | | | | | |
| at 72 hrs | | | | | | | | | |
| (ng/cm²) | | | | | | | | | |
| Flux | 800 | 400 | 300 | 70 | 70 | 400 | 500 | 400 | 400 |
| (ng/cm²/ | | | | | | | | | |
| hr) | | | | | | | | | |

### Example 15

Additional synthetic delta-9-THC formulations for transdermal delivery (Formulation Nos. 028 to 034) were prepared by mixing ingredients as shown in Table 9:

TABLE 9
Transdermal Synthetic delta-9-THC formulation nos. 028 to 034

| | 028 | 029 | 030 | 031 | 032 | 033 | 034 |
|---|---|---|---|---|---|---|---|
| Ingredients | (% W/V) | (% W/V) | (% W/V) | (% W/V) | (% W/V) | (% W/V) | (% W/V) |
| THC | 2.0 | 2.0 | 2.0 | 2.0 | 2.0 | 2.0 | 2.0 |
| Ethanol | 42.4 | 42.4 | 42.4 | 42.4 | 42.4 | 42.4 | 42.4 |
| PG | 27.8 | 27.8 | 27.8 | 27.8 | 27.8 | 27.8 | 27.8 |
| 1,3 Butanediol | 27.8 | | | | | | |
| Dipropylene Glycol | | 27.8 | | | | | |
| Hexylene Glycol | | | 27.8 | | | | |
| PEG-400 | | | | 27.8 | | | |
| Tween-20 | | | | | 27.8 | | |
| Tween-80 | | | | | | 27.8 | |
| Span-20 | | | | | | | 27.8 |

Synthetic delta-9-THC formulations for transdermal delivery (formulation nos. 028 to 034) were prepared by the same procedure described in Example 12. Flux measurement was also performed as described in Example 12. The experimental conditions are the same as provided in Table 3 of Example 12.

Flux of THC through the human cadaver skin was measured for a minimum period of 120 hours (5 days) and results of the flux measurement experiments are provided in Table 10.

TABLE 10
Transdermal Synthetic delta-9-THC formulations

| | Formulation Nos. | | | | | | |
|---|---|---|---|---|---|---|---|
| | 028 | 029 | 030 | 031 | 032 | 033 | 034 |
| Total Amount of THC | 103400 | 86400 | 115300 | 61400 | 7400 | 30100 | 43800 |
| Permeated at 120 Hrs | (9%) | (17%) | (53%) | (43%) | (80%) | (137%) | (47%) |
| (ng/cm²) | | | | | | | |
| Flux (ng/cm²/hr) | 900 | 700 | 1000 | 500 | 60 | 300 | 400 |

### Example 16

Additional synthetic delta-9-THC formulations for transdermal delivery patches (Formulation Nos. 035 to 038) were prepared by mixing ingredients as shown in Table 11:

TABLE 11
Transdermal Synthetic delta-9-THC

| | 035 | 036 | 037 | 038 |
|---|---|---|---|---|
| Ingredients | (% W/W) | (% W/W) | (% W/W) | (% W/W) |
| THC | 2.0 | 2.0 | 2.0 | 2.0 |
| PG | 27.8 | 27.8 | 27.8 | 27.8 |
| Hexylene Glycol | 27.8 | 27.8 | 27.8 | 27.8 |
| Durotak 9301 | 42.4 | | | |
| Durotak 2516 | | 42.4 | | |
| Durotak 2207 | | | 42.4 | |
| Silicone Adhesive | | | | 42.4 |

To prepare a transdermal patch containing synthetic delta-9-THC, all of the components from Table 11, with the exception of the THC, were mixed together with stirring for 18 hours. Next, the THC was added 30 minutes before spreading the formulation. The formulation was spread using a commercial benchtop spreader. Specifically, the formulation matrix is evenly spread onto an 8×14 inch sheet of release liner (such as 3M 9744) to a thickness of 0.5 mm The sheet is then place in an oven at 100° F. for one hour to evaporate off the ethyl acetate and ethanol adhesive solvent. An opaque backing membrane (such as 3M 9730 NR film) with low permeability to oxygen, for inhibition of photo and oxidative degradation, is then carefully applied to the sheet by hand to avoid formation of bubbles and voids. A circular die (1.5 inches diameter) was used to cut patches (7 cm²) for subsequent studies.

The general procedure for flux measurements of transdermal formulations in the examples above was as follows. The human cadaver skin, stored at −80° C., was thawed at room temperature in PBS, and visually inspected for defects before use. Transdermal flux was measured using standard Franz diffusion cells composed of a cylindrical donor compartment and a separate water jacketed cylindrical receptor compartment with the volume of 13 mL. The human cadaver skin was clamped between the two compartments with the dermis side facing toward the receptor compartment. The general procedure for flux measurement of the transdermal adhesive patch is as follows. The release liner is peeled off the patch and the adhesive surface is applied to a piece of human cadaver skin (Example 16, Table 11 only). The transdermal patch was adhered to the skin with the patch on the side of the skin in contact with the donor compartment. The receptor compartment was filled with receptor medium, held at constant temperature, and constantly stirred to collect the THC as it diffuses from the adhered patch, through the skin and into receptor compartment. It was confirmed that the receptor fluid was always in contact with the skin. The receptor compartment was emptied at 24 hour intervals for assay of delta-9-THC and replaced with fresh receptor solution. In order to maintain the sink condition in receptor compartment, the delta-9THC concentration in the receptor compartment was maintained at less than 10% of its solubility. The experimental conditions are the same as provided in Table 3 of Example 12.

### Example 17

Synthetic delta-9-THC (THC) formulations for transdermal delivery (Formulation Nos. 040 through 042) were prepared by mixing ingredients as shown in Table 12:

TABLE 12
Transdermal Synthetic delta-9-THC formulation no. 040 to 042

| | THC-040 | THC-041 | THC-042 |
|---|---|---|---|
| Excipients | (Solution) | (Solution) | (Gel) |
| THC in ethanol (20% w/w) | 13.1% | 12.8% | 13.6% |
| Ethanol | 43.2% | 38.6% | 41.9% |
| Propylene Glycol | 43.7% | 38.6% | 43.4% |
| Hydramol | — | 10.0% | — |
| Klucel HF | — | — | 1.0% |

The following examples used composition THC_040 as an example for preparing a transdermal patch. The above excipients except THC were mixed together by stirring for 18 hours and then the THC in ethanol was added into the above excipient mixture.

The general procedure for flux measurement of transdermal formulation was conducted as follows. The human cadaver skin, stored at −80 C, was thawed at room temperature in PBS, and visually inspected for defects before using in the study. Transdermal flux was measured using standard Franz diffusion cells composed of a cylindrical donor compartment and a separate water jacketed cylindrical receptor compartment with the volume of 13 ml. The human cadaver skin was clamped between the two compartments with the dermis side facing toward the receptor compartment. The receptor compartment was filled with receptor medium, held at constant temperature, and constantly stirred to collect the THC as it diffuses through the skin and into receptor compartment, making sure that that the receptor fluid was always in contact with the skin. The receptor compartment was emptied at 24 hr intervals for assay of delta-9-THC and replaced with fresh receptor solution. In order to maintain the sink condition in receptor compartment, the delta-9-THC concentration in receptor compartment was kept at less than 10% of its solubility. The experimental conditions were as follows:

Experimental Condition for In-vitro Permeability testing

| | De-ionize water + 0.5% |
|---|---|
| | Brij-O(20) + 0.01% |
| Receiving Media | Sodium Azide |
| Receiving Media Volume (ml) | 13 |
| Sample Volume (ml) | 13 |
| Sampling Interval (hr) | 24, 48, 72, 96, 120 |
| Franz-cell diffusion area (sqcm) | 1.76 |
| Membrane Type | Human Cadaver Skin |

TABLE 13
Flux is measure for a minimum period of 120 Hrs (5 days)

| | THC-040 | THC-041 | THC-042 |
|---|---|---|---|
| Avg Flux 0-24 hr, μg/sqcm/hr | 0.11 | 0.00 | 0.00 |
| (% RSD) | | | |
| Avg Flux 24-48 hr, μg/sqcm/hr | 0.72 | 0.59 | 2.16 |
| (% RSD) | (173.21) | (62.53) | (62.29) |
| Avg Flux 48-72 hr, μg/sqcm/hr | 1.55 | 0.83 | 2.93 |
| (% RSD) | (50.46) | (12.42) | (33.55) |
| Avg Flux 72-96 hr, μg/sqcm/hr | 2.15 | 0.66 | 3.03 |
| (% RSD) | (49.90) | (39.91) | (10.27) |
| Avg Flux 96-120 hr, μg/sqcm/hr | 1.66 | 0.22 | 2.03 |
| (% RSD) | (39.69) | (87.14) | (10.04) |
| Avg Flux 24-120 hr, μg/sqcm/hr | 1.24 | 0.46 | 2.03 |
| (% RSD) | (97.00) | (40.40) | (29.04) |

### Example 18

Synthetic delta-9-THC (THC) formulations for transdermal delivery (Formulation Nos. 043 through 0048) were prepared by mixing ingredients as shown in Table 14:

TABLE 14
THC Matrix System

| | THC | THC | THC | THC | | |
|---|---|---|---|---|---|---|
| Excipients | 043-R1 | 044-R1 | 045-R1 | 046 | THC 047 | THC 048 |
| THC | 4.05% | 4.05% | 4.05% | 7.81% | 7.86% | 7.79% |
| Oleic acid | — | 4.45% | — | — | — | — |
| Propylene Glycol | 17.00% | 12.55% | 12.16% | — | 14.94% | 15.19% |
| Isopropyl Palmitate | — | — | 4.46% | — | — | — |
| DMSO | — | — | — | 15.17% | — | — |
| Lactic Acid | — | — | — | 14.85% | 15.92% | 14.86% |
| GMO | — | — | — | 4.84% | 4.87% | 4.95% |
| Hydramol | — | — | — | 5.49% | 5.20% | 5.28% |
| BIO-PSA 4501 | 78.95% | 78.95% | 79.32% | — | — | — |
| DURO-TAK 9301 | — | — | — | 51.84% | 51.21% | — |
| DURO-TAK 2054 | — | — | — | — | — | 51.92% |

The above ingredients (Table 14) were blended by stirring for 18 hours and then, using a commercial benchtop spreader, the matrix was evenly spread onto an 8×14 inch sheet of release liner (such as 3M 9744) to a thickness of 0.5 mm The sheet was then placed in an oven at 86 F for 120 min to evaporate off the ethyl acetate adhesive solvent. An opaque backing membrane (such as 3M 9730 NR film) with low permeability to oxygen to inhibit photo and oxidative degradation, was then carefully applied by hand to avoid formation of bubbles and voids. A circular die (1.5 inches diameter) was used to cut patches (1.76 sqcm) for subsequent studies. After drying, the drug adhesive matrix had a surface density of 2-30 mg/sqcm, containing THC in 5-20% w/w.

The prepared transdermal formulations were then subjected to a flux measurement test as follows. Human cadaver skin, stored at −80° C., was thawed at room temperature in phosphate buffered saline (PBS), and visually inspected for defects before using it in the study. Transdermal flux was then measured using standard Franz diffusion cells composed of a cylindrical donor compartment and a separate water jacketed cylindrical receptor compartment with the volume of 13 mL. The human cadaver skin was clamped between the two compartments with the dermis side facing toward the receptor compartment. The donor compartment was filled with the transdermal THC formulations prepared as described above. The receptor compartment was filled with receptor medium, held at constant temperature, and constantly stirred to collect the THC as it diffuses through the skin and into receptor compartment, making sure that the receptor fluid was always in contact with the skin. The receptor compartment was emptied at 24 hr intervals for assay of THC and replaced with fresh receptor solution. In order to maintain the sink condition in receptor compartment, the THC concentration in receptor compartment was kept at less than 10% of its solubility. The experimental conditions are provided in Table 15:

TABLE 15
Experimental Condition for In-vitro Permeability testing

| | De-ionized water + 0.5% |
|---|---|
| | Brij-O(20) + 0.01% |
| Receiving Media | Sodium Azide |
| Receiving Media Volume (mL) | 13 |
| Sample Volume (mL) | 13 |
| Sampling Interval (hr) | 24, 48, 72, 96, 120, 144 |
| Franz-cell diffusion area (sqcm) | 1.76 |
| Membrane Type | Human Cadaver Skin |

Flux of THC through the human cadaver skin was measured for a minimum period of 144 Hrs (6 days) and results of the flux measurement are provided in Table 16.

TABLE 16
THC Flux Results

| | THC | THC | THC | THC | THC | THC |
|---|---|---|---|---|---|---|
| | 043-R1 | 044-R1 | 045-R1 | 046 | 047 | 048 |
| Avg Flux 0- | 0.00 | 0.28 | 0.00 | 0.00 | 0.00 | N/A |
| 24 hr, μg/sqcm/hr (% RSD) | | (173.21) | | | | |
| Avg Flux 24- | 0.00 | 1.01 | 0.00 | 0.00 | 0.00 | N/A |
| 48 hr, μg/sqcm/hr (% RSD) | | (16.39) | | | | |
| Avg Flux 48- | 0.00 | 0.96 | 0.52 | 0.00 | 0.00 | N/A |
| 72 hr, μg/sqcm/hr (% RSD) | | (12.86) | (82.26) | | | |
| Avg Flux 72- | 0.00 | 1.07 | 0.84 | 0.00 | 0.00 | N/A |
| 96 hr, μg/sqcm/hr (% RSD) | | (13.81) | (2.82) | | | |
| Avg Flux 96- | 0.00 | 1.22 | 0.94 | 0.00 | 0.00 | N/A |
| 120 hr, μg/sqcm/hr (% RSD) | | (15.51) | (2.75) | | | |
| Avg Flux 120- | 0.00 | 1.03 | 0.56 | 0.00 | 0.00 | N/A |
| 144 hr, μg/sqcm/hr (% RSD) | | (12.46) | (86.67) | | | |
| Avg Flux 24- | 0.00 | 0.93 | 0.43 | 0.00 | 0.00 | N/A |
| 144 hr, μg/sqcm/hr (% RSD) | | (21.54) | (33.55) | | | |

### Example 19

| Ingredients | THC_061 | THC_059 | THC_057 |
|---|---|---|---|
| Synthetic Delta 9-THC | 5.0% | 5.00% | 5.00% |
| Span 20 (Croda) | 7.50% | | |
| Crodamol OO | | 7.50% | |
| Lauryl Lactate | | | 7.50% |
| BIO PSA 4501 | 87.50% | 87.50% | 87.50% |

| | |
|---|---|
| Process Solvent | 200 proof ethanol (as THC ethanolic solution), |
| | Heptane (inadhesive BIO-PSA 4501) |

| | THC_061 | THC_059 | THC_057 |
|---|---|---|---|
| Total Amt of THC permeated | 37010 | 40290 | 41800 |
| at 168 hrs (ng/cm2) | | | |
| Flux (ng/cm²/hr) | 220 | 240 | 249 |
| Calculated Patch area cm² | 30 | 25 | 25 |
| equivalent to approx. 2.5 | | | |
| mg/day oral dose | | | |
| Calculated Patch area cm² | 50 | 45 | 45 |
| equivalent to approx. | | | |
| 5 mg/day oral dose | | | |

### Example 20

The effect of gelling agents and their concentration on the permeation of THC through human cadaver skin. THC gel formulation can be gelled by gelling agents including but not limited to, natural polymers such as natural polymers, polysaccharides and its derivatives such as but not limited to (agar, alginic acid and derivatives, cassia tora, collagen, gelatin, gellum gum, guar gum, pectin, potassium or sodium carrageenan, tragacanth, xanthum gum, copal, starch, chitosan, resin etc.), synthetic polymers and its derivatives such as without any limitation to carboxyvinyl polymers or carbomers (carbopol 940, carbopol 934, carbopol 971), polyethylene and its copolymers etc. clays such as silicate etc. polyvinyl alcohol, polyacrylamide, polyvinyl pyrrolidone homopolymer and polyvinyl pyyrolidone copolymers (PVP, Poloxamer), acrylic acid its ester, polyacrylate copolymers, isobutylene, ethylene vinyl acetate copolymers, natural rubbers, synthetic rubbers such as styrene-diene copolymers, styrene-butadiene block copolymers, isoprene block copolymers, acrylonitrile butadiene rubber, butyl rubber or neoprene rubber, as well as pressure sensitive adhesive based on silicone, or “hot-melt adhesive”. In addition, other than human cadaver skin, clobazam can be evaluated with other artificial membranes including but not limited to cellulose membrane, silicone membranes (polydimethylsiloxane), liposome coated membranes, solid-supported liquid membranes, lecithin organogel membrane and other. Besides the gel formulation of clobazam, other dosage forms including but not limited to ointment, creams, emulsion, liposomes, etc. may be used.

### Example 21

The effect of enhancers or solubilizers on the flux of clobazam through human cadaver skin was evaluated. The desire optimum composition of clobazam gel formulation contained dimethylsulfoxide (DMSO), dimethylisosorbide (DMI), Lactic acid, Tween-20, highly purified diethylene glycol monoethyl ether (Transcutol P), dipropylene glycol, polyethylene glycol-400, propylene glycol (PG), Hexylene Glycol (HG), Lauroglycol-90. Apart from above mentioned enhancers and/or solubilizers, the clobazam transdermal delivery can be influenced by enhancers and/or solubilizers including but not limited water, sulfoxides, and similar chemicals such as but not limited to (dimethylsulfoxide, dimethylacetamide, dimethylformamide, decylmethylsulfoxide, dimethylisosorbide etc), azone, pyrrolidones such as but not limited to (N-methyl-2-pyrrolidone, 2-pyrrolidon etc), esters such as but not limited to (Propylene glycol monolaurate, butyl ethanoate, ethyl ethanoate, isopropyl myristate, isopropyl palmitate, methyl ethanoate, decyl oleate, glycerol monooleate, glycerol monolaurate, lauryl laurate etc), fatty acids such as but not limited to (capric acid, caprylic acid, lauric acid, oleic acid, myristic acid, linoleic acid, stearic acid, palmitic acid etc), alcohols, fatty alcohols and glycols such as but not limited to (oleyl alcohol, nathanol, dodecanol, propylene glycol, glycerol etc), ethers such as but not limited to (diethylene glycol monoethyl ether), urea, polyoxyethylene fatty alcohol ethers, polyoxyethylene fatty acid esters, esters of fatty alcohols, esters of long chain fatty acids with methyl, ethyl or isopropyl alcohol, esters of fatty alcohols with acetic acid, lactic acid, as well as oleic acid diethanolamine, essential oils, terpene and terpenoids such as but not limited to (terpineol, limonene, thymol, cineole etc), surfactant type enhancers (polysorbate 80, polysorbate 20 etc.), liposomes, niosomes, transferomes, ethanosomes, polysorbate such as but not limited to (polysorbate 20, polysorbate 40, polysorbate 60, polysorbate 80 etc), span such as but not limited to (span 80, span 20 etc), surfactants such as (anionic, cationic, nonionic and amphoteric), propylene glycol monocaprylate type I, propylene glycol monocaprylate type II, propylene glycol dicaprylate, medium chain triglycerides, propylene glycol monolaurate type II, linoleoyl polyoxyl-6 glycerides, Caprylic glyceride, oleoyl-polyoxyl-6-glycerides, lauroyl polyoxyl-6-gylcerides, polyglyceryl-3-dioleate, diethylene glycol monoethyl ether, propylene glycol monolaurate type I etc, cyclodextrins, polyhydric alcohol, especially 1,2-propanediol, butanediol, glycerine, polyethylene glycol (m.w. 100 and higher), Dimethyl Sulfoxide, Dimethyl Isosorbide, tetrahydrofurfuryl alcohol, diethyl tolumide, monoisopropylidene glycerine and others Solubilizers, surfactants, emulsifying agents, dispersing agents and similar compounds or chemicals known to those skilled in the art can be used either alone or in combination thereof.

### Example 22

Oral bioavailability of THC is only 10-20%. For our calculation purpose, we took an average bioavailability of 15% 17. Accordingly, the actual dose delivering to patient upon oral delivery is described in Table 17.

TABLE 17
Theoretical dose required from Transdermal Dosage form.

| Oral Dose | Transdermal Dose range (g/day) |
|---|---|
| 5 mg/day | 0.75 |
| 10 mg/day | 1.5 |
| 20 mg/day | 3 |

$$\begin{matrix}Flux Required = Dose / Surface area \\ = 3. mg / day / surface area \\ = 3000 ug / 24 hr / 50 sqcm \\ = 2.5 ug / sqcm / hr\end{matrix}$$

Accordingly, 50 sqcm patch with 2.5 ug/sqcm/hr flux will deliver 3 mg of drug in one day through transdermal route which is equivalent to 20 mg/day oral dose. According to table, The maximum dose of approved dronabinol is 20 mg/day which is feasible to deliver through this formulation.

1. A method for the treatment of a neurological disease or disorder comprising:
a. selecting a patient in need of treatment of a neurological disease or disorder;
b. topically applying:
i. a pharmaceutical composition in the form of a transdermal patch comprising: dronabinol, wherein the concentration of the dronabinol is from about 0.1% to about 15% w/w; wherein the pharmaceutical composition further comprises:
ii. about 1% to about 17% w/w of a solvent;
iii. about 1% to about 25% w/w of a penetration enhancer;
iv. about 35% to about 80% w/w of a pressure sensitive adhesive such as a silicone pressure sensitive adhesive, polyisobutylene adhesive;
v. optionally a polymer and/or a stabilizer such as an antioxidant,
wherein the neurological disease or disorder is selected from the group consisting of dementia, amyotrophic lateral sclerosis (ALS), frontotemporal dementia (FTD), ALS with FTD, Alzheimer's disease (AD), Parkinson's disease (PD), Huntington's disease, progressive supranuclear palsy (PSP), brain trauma, spinal cord injury, corticobasal degeneration (CBD), nerve injuries, neuropathies, and TDP43 proteinopathies, chronic traumatic encephalopathy, Perry Syndrome, Dementia with Lewy body in association with Alzheimer's disease, Parkinson's disease with or without dementia, and Limbic-predominant age-related TDP-43 encephalopathy (LATE).

2. The method of claim 1, wherein dronabinol is in a form selected from the group consisting of co-crystals, amorphous, coated, crystalline, a salt, an isomer, a solid solution, a prodrug, an analog, a derivative, a metabolite, a solution, synthetic, an ethanol solution, sesame oil solution and a naturally derived delta-9-tetrahydrocannabinol.

3. The method of claim 1, wherein dronabinol is selected from a group consisting of amorphous dronabinol, crystalline dronabinol, co-crystals of dronabinol, coated dronabinol, and ethanolic solution, sesame oil solution of dronabinol or/and any vegetable oil of dronabinol in the range of 0.01%-95% w/w or w/v.

4. The method of claim 1, wherein dronabinol is in neat dronabinol or ethanolic solution of dronabinol or sesame oil solution of dronabinol.

5. The method of claim 1, wherein the pharmaceutical composition in the form of a transdermal patch comprises a carrier or an ingredient in effective amount either alone or in combinations thereof selected from the group consisting of solvents, cosolvents, gelling agents, polymers, penetration enhancers, emollients, skin irritation reducing agents, buffering agents, pH stabilizers, solubilizers, suspending agents, dispersing agents, stabilizers, plasticizers, surfactants, emulsifiers, antioxidants, and oxidants.

6. The method of claim 1, wherein the pharmaceutical composition in the form of a transdermal patch is formulated as microneedles.

7. The method of claim 1, wherein the topical application of a transdermal patch for the treatment of a neurological disease or disorder is selected from the group consisting of once in a day, once in two days, once in three days, once in four days, once in five days, once in six days, once in a week, once in ten days.

8. The method of claim 1, wherein the transdermal patch is in the form of a transdermal matrix patch.

9. The method of claim 1 wherein the transdermal patch is in the form of a transdermal reservoir patch wherein reservoir formulation is a liquid formulation or a semisolid formulation and formulation does not contain a pressure sensitive adhesive.

10. The method of claim 9, wherein the pharmaceutical composition in the form of a transdermal reservoir formulation and comprises a carrier or an ingredient in effective amount either alone or in combinations thereof selected from the group consisting of solvents, cosolvents, gelling agents, polymers, penetration enhancers, emollients, skin irritation reducing agents, buffering agents, pH stabilizers, solubilizers, suspending agents, dispersing agents, stabilizers, plasticizers, surfactants, antioxidants, and oxidants.

11. The method of claim 1 further providing a constant rate of delivery of the active components of the transdermal patch over a time period.

12. The method of claim 1 further providing a steady absorption rate of the active components of the transdermal patch over a time period.

13. The method of claim 1 further achieving a constant blood serum level of the active components of the transdermal patch over a time period.

14. The method of claim 1 further achieving a reduced variability in dosage of the active components of the transdermal patches over a time period.

15. The method of claim 1 further providing a plasma concentration of the active components of the transdermal patch in a therapeutic range over a period of time.

16. The method of claim 1, wherein the neurological disease or disorder is Dementia.

17. The method of claim 1, wherein the neurological disease or disorder is Alzheimer's Disease.

18. The method of claim 1, wherein the composition is a liquid formulation and/or a semisolid formulation, wherein the topically applying is done two to six times in a day, once in a day, once in two days, once in three days, once in four days, once in five days, once in six days, once in a week.

19. A method for the treatment and/or prevention and/or control of seizure disorder in a patient comprising:
a. selecting a patient in need of treatment and/or prevention and/or control of seizure disorder;
b. topically applying:
i. a pharmaceutical composition in the form of a transdermal patch comprising: dronabinol, wherein the concentration of the dronabinol is from about 0.1% to about 15% w/w; wherein the pharmaceutical composition further comprises:
ii. about 1% to about 17% w/w of a solvent;
iii. about 1% to about 25% w/w of a penetration enhancer;
iv. about 35% to about 80% w/w of a silicone pressure sensitive adhesive; at least one suspending agent comprising silicon dioxide;
v. an antioxidant comprising BHT,
wherein the transdermal patch provides an average flux of the dronabinol of about 0.43 to about 0.93 μg/cm²/hr over at least 6 days.

20. The method of claim 19, wherein the seizure disorder includes complex partial seizures, simple partial seizures, partial seizures with secondary generalization, generalized seizures (including absence, grand mal (tonic clonic), status epilepticus, tonic, atonic, myoclonic), neonatal and infantile spasms, drug-induced seizures, trauma-induced seizures, and febrile seizures, and additional specific epilepsy syndromes such as juvenile myoclonic epilepsy, Lennox-Gastaut, Dravet syndrome, Tuberous Sclerosis Complex (TSC), Treatment-Resistant Epilepsy, Treatment Resistant Pediatric Epilepsy, mesial temporal lobe epilepsy, nocturnal frontal lobe epilepsy, progressive epilepsy with mental retardation, and progressive myoclonic epilepsy, as well as seizures associated with CNS mass lesions.

21. The method of claim 19 wherein the topical application of a transdermal patch for the treatment and/or prevention and/or control of seizure disorder in a patient, wherein the seizure disorder include, for example, complex partial seizures, simple partial seizures, partial seizures with secondary generalization, generalized seizures (including absence, grand mal (tonic clonic), status epilepticus, tonic, atonic, myoclonic), neonatal and infantile spasms, drug-induced seizures, trauma-induced seizures, and febrile seizures, and additional specific epilepsy syndromes such as juvenile myoclonic epilepsy, Lennox-Gastaut, Dravet syndrome, Tuberous Sclerosis Complex (TSC), Treatment-Resistant Epilepsy, Treatment Resistant Pediatric Epilepsy, mesial temporal lobe epilepsy, nocturnal frontal lobe epilepsy, progressive epilepsy with mental retardation, and progressive myoclonic epilepsy, as well as seizures associated with CNS mass lesions is selected from the group consisting of once in a day, once in two days, once in three days, once in four days, once in five days, once in six days, once in a week, once in ten days.

22. The method of claim 19, wherein the transdermal patch is in the form of a transdermal matrix patch.

23. The method of claim 19 further providing a constant rate of delivery of the active components of the transdermal patch over a time period.

24. The method of claim 19 further providing a steady absorption rates of the active components of the transdermal patch over a time period.

25. The method of claim 19 further achieving a constant blood serum levels of the active components of the transdermal patch over a time period.

26. The method of claim 19 further achieving a reduced variability in dosage of the active components of the transdermal patches over a time period.

27. The method of claim 19 further providing a plasma concentration of the active components of the transdermal patch in a therapeutic range over a period of time.

28. A method of treating one or more symptoms of a psychological disorder in a subject comprising:
a. selecting a patient in need of treating one or more symptoms of a psychological disorder;
b. topically applying:
i. a pharmaceutical composition in the form of a transdermal patch comprising: dronabinol, wherein the concentration of the dronabinol is from about 0.1% to about 15% w/w; wherein the pharmaceutical composition further comprises:
ii. about 1% to about 17% w/w of a solvent;
iii. about 1% to about 25% w/w of a penetration enhancer;
iv. about 35% to about 80% w/w of a silicone pressure sensitive adhesive; at least one suspending agent comprising silicon dioxide;
v. an antioxidant comprising BHT,
wherein the transdermal patch provides an average flux of the dronabinol of about 0.43 to about 0.93 μg/cm²/hr over at least 6 days,
wherein the subject has symptoms of at least one of agitation, agitation associated with CTE, anxiety, stress, insomnia, and/or depression when assessed by a validated scale or biomarker for identifying symptoms of agitation, agitation associated with CTE, anxiety, stress, insomnia, and/or depression.

29. The method of claim 28 wherein the subject has symptoms of two or more of agitation, agitation associated with CTE, anxiety, stress, insomnia, and/or depression.

30. The method of claim 28, wherein the transdermal patch is in the form of a transdermal matrix patch.

31. The method of claim 28 wherein the method: i. reduces the subject's score on the validated scale for anxiety or reduces the agitation, agitation associated with CTE, anxiety biomarker; ii. reduces the subject's score on the validated scale for stress or reduces the stress biomarker; iii. reduces the subject's score on the validated scale for depression or the depression biomarker; and/or reduces the subject's score on the validated scale for insomnia or reduces an insomnia biomarker.

32. A pharmaceutical composition in the form of a transdermal patch comprising:
a. dronabinol, wherein the concentration of the dronabinol is from about 0.1% to about 15% w/w; wherein the pharmaceutical composition further comprises:
b. about 1% to about 17% w/w of a solvent;
c. about 1% to about 25% w/w of a penetration enhancer;
d. about 35% to about 80% w/w of a silicone pressure sensitive adhesive; at least one suspending agent comprising silicon dioxide;
e. an antioxidant comprising BHT.

33. The pharmaceutical composition of claim 32, wherein the transdermal patch is in the form of a transdermal matrix patch.

34. The pharmaceutical composition of claim 32, wherein the transdermal patch is in the form of a transdermal reservoir patch wherein reservoir formulation is a liquid formulation or a semisolid formulation and formulation does not contain a pressure sensitive adhesive.

35. The pharmaceutical composition of claim 32, wherein the pharmaceutical composition in the form of a transdermal reservoir formulation and comprises a carrier or an ingredient in effective amount either alone or in combinations thereof selected from the group consisting of solvents, cosolvents, gelling agents, polymers, penetration enhancers, emollients, skin irritation reducing agents, buffering agents, pH stabilizers, solubilizers, suspending agents, dispersing agents, stabilizers, plasticizers, surfactants, antioxidants, and oxidants.
